(19) United States
(12) Patent Application Publication
(10) Pub. No.: US 2022/0218850 A1
(43) Pub. Date: Jul. 14, 2022

(54) Methods and Compositions for Two-Stage Microbubble Delivery of Active Agents

(71) Applicant: Virginia Commonwealth University, Richmond, VA (US)

(72) Inventors: Paul B. Fisher, Richmond, VA (US); Luni Emdad, Richmond, VA (US); Praveen Bhoopathi, Richmond, VA (US); Anjan K. Pradhan, Richmond, VA (US); Swadesh K. Das, Richmond, VA (US); Alexander L. Klibanov, Charlottesville, VA (US)

(21) Appl. No.: 17/574,790

(22) Filed: Jan. 13, 2022

Related U.S. Application Data
(60) Provisional application No. 63/136,675, filed on Jan. 13, 2021.
(60) Provisional application No. 63/209,178, filed on Jun. 10, 2021.

(51) Int. Cl. A61K 49/22; A61K 45/06; A61K 31/7115

(52) U.S. Cl. CPC A61K 49/223; A61K 31/7115; A61K 45/06

(57) ABSTRACT

In various aspects, the present disclosure provides methods, compositions, and kits for treating a target tissue with one or more active agents. In embodiments, delivering an active agent to a target tissue comprises administration of microbubbles and ultrasound.

### SEQUENCE LISTING

The instant application contains a Sequence Listing which has been submitted electronically in ASCII format and is hereby incorporated by reference in its entirety. Said ASCII copy is written in the file named 053151-516001WO_ST25.txt, which was created on Jan. 9, 2022, and is 18,298 bytes in size.

### BACKGROUND

Cancer is a leading cause of death and is responsible for increasing health costs. Traditionally, cancer has been treated using chemotherapy, radiotherapy and surgical methods. Tumor cell plasticity and heterogeneity, however, remain challenges for effective treatments of many cancers. In addition, traditional therapies may have drawbacks, e.g. insufficient specificity, intolerable toxicity and too low efficacy. Challenges may also arise when attempting to direct treatments to particular tissues, and especially tissues that are more difficult to target, such as the brain. Moreover, such challenges are not limited to treatment of cancer, where inefficient targeting to a tissue of interest may limit efficacy and/or increase side effects of therapeutic agents, or limit diagnostic methods using imaging agents.

### BRIEF SUMMARY

In view of the foregoing, there is a need for improved targeting of active agents (including therapeutic and imaging agents, and for “theranostic” applications where both a therapeutic agent and imaging agent are delivered at the same time), as well as improved targeting in cancer therapies. The present disclosure provides methods and compositions that address this need, and provide additional benefits as well.

In some aspects, the present disclosure provides a method of administering an active agent to a target tissue. In embodiments, the active agent is a therapeutic agent or an imaging agent. In embodiments, the method includes: (a) administering to a subject a first microbubble composition, the first microbubble composition containing first microbubbles and not containing the active agent; (b) a first ultrasound administration directed to the target tissue that disrupts the first microbubbles; (c) administering to the subject a second microbubble composition after the first ultrasound administration, the second microbubble composition containing second microbubbles complexed with the active agent; and (d) a second ultrasound administration directed to the target tissue that disrupts the second microbubbles and releases the active agent to the target tissue.

In embodiments, methods described herein are referred to as focused ultrasound (FUS) double microbubble (FUS-DMB) delivery. In embodiments, the FUS-DMB is applied to treat brain cancers, such as those developing in the brain (e.g., glioblastoma multiforme, or GBM) as well as metastatic tumors in the brain with primary tumor sites outside the brain. In embodiments, FUS-DMB involves first transiently opening the blood brain barrier (BBB) using microbubbles (MBs) lacking the active agent to be delivered (e.g., empty MBs) using focused ultrasound at a target tissue, and then application of an ultrasound-targeted microbubble-destruction (UTMD) technique in which the active agent (e.g., adenovirus, or therapeutic proteins) is complexed with microbubbles that are systemically (or directly) administered and released in the target tissue (e.g., brain or pancreas) by FUS. In embodiments, advantages include treatment of primary GBM, recurrent GBM, or secondary brain tumors (resulting from metastasis from other sites in the body), without a need for surgery. In embodiments, active agent (e.g., viruses) complexed with MBs added directly to surgically debulked tumors and application of FUS is used to enhance therapeutic activity.

In some aspects, the present disclosure provides a kit for use in the treatment of a target tissue with an active agent. In embodiments, the kit includes a first and second microbubble composition, wherein (i) the active agent is a therapeutic agent or an imaging agent, (ii) the first microbubble composition contains first microbubbles and does not contain the active agent, and (iii) the second microbubble composition contains second microbubbles complexed with the active agent.

In embodiments, the use of the kit includes: (a) administration of the first microbubble composition, (b) a first ultrasound administration directed to the target tissue that disrupts the first microbubbles, (c) administration of the second microbubble composition after the first ultrasound administration, and (d) a second ultrasound administration directed to the target tissue that disrupts the second microbubbles.

### BRIEF DESCRIPTION OF THE DRAWINGS

FIGS. 1A-1C show a focused ultrasound (FUS) dual microbubble (FUS-DMB) delivery strategy for delivering adenoviruses in the brain, in accordance with an embodiment. FIG. 1A provides a schematic representation of a FUS-DMB delivery protocol. Briefly, 100 μl of diluted microbubbles (MBs)+Ad.5/3-CMV-Luc were injected through the tail vein and allowed to circulate for 15 sec. After 15 sec, mouse brains were sonicated (using focused ultrasound (FUS) for 1 min (Group 1; left panel FIG. 1B and 1^{st} bar of FIG. 1C). The second group (Group 2; right panel FIG. 1B and 2^{nd} bar of FIG. 1C) were injected with diluted microbubbles (empty) and sonicated for 1 min (FUS) in the brain region and then injected I.V. with 100 μl MBs containing Ad.5/3-CMV-Luc and the brain region was sonicated (FUS) for 1 min after allowing the MBs to circulate for 15 sec (a FUS-DMB delivery approach). The next day these animals were imaged using an IVIS imager. FIG. 1B shows representative photographs. FIG. 1C shows relative luciferase intensity as measured in individual animals at a single time point, and the average value from three mice is plotted. *Statistically significant.

FIGS. 2A and 2B show Focused Ultrasound (FUS) Dual MB (FUS-DMB) Delivery, and efficient delivery of therapeutic viruses in the brain, in accordance with an embodiment. FIG. 2A shows a schematic of an example experimental protocol. Mice were anesthetized via intraperitoneal (I.P.) administration of ketamine (40 mg/kg) and xylazine (3 mg/kg) and immobilized in a stereotactic frame. Intracerebral injection of 10,000 glioma cells (GBM-6-Luc) in 5 μl of DMEM medium was performed over 10 minutes using a Hamilton syringe. The skull opening was closed using sterile bone wax, and the skin incision was closed using sterile surgical staples or surgical glue. A week later these mice were imaged and randomized into 3 groups based on tumor growth (IVIS Image). These tumors were treated by intravenous (I.V.) injections of microbubbles (MBs) containing either Ad.5/3-Null or Ad.5/3-CTV with or without BBB opening (using FUS). FIG. 2B shows representative photographs (upper panel), and relative luciferase intensity measurements (lower panel) in individual animals at a single time point and the average value from three mice is plotted. *Statistically significant.

FIG. 3A shows images of a preparation of His-MDA-7 protein in complex with microbubbles (MBs) (MDA-7-MB). To confirm the association of His-MDA-7 with MBs, Alexa Fluor 488 labeled His-MDA-7 was mixed and incubated overnight with lyophilized MBs. The unincorporated labeled His-MDA-7 was removed by centrifugation and the MBs (white layer) was mixed with 1 ml PBS and observed under a fluorescent microscope. Scale; 1 μm. The labeled His-MDA-7 (identified by green fluorescence) was associated with the lipid shell of the MBs.

FIG. 3B shows tumor specific delivery of Alexa Fluor-His-MDA-7 encapsulated MBs coupled with ultrasound targeted MB destruction (UTMD). DU-145 human prostate cancer cells were established as xenografts in the left flank of nude mice. Alexa Fluor labeled His-MDA-7/MBs complex were injected through the tail vein of nude mice, and sonoporated in the xenograft (DU-145) tumor implanted in the left flank with a portable ultrasound (SonoSite Micro-Maxx US platform) equipped with an L25 linear array transducer set at 0.7 Mechanical Index, 1.8 MPa for 10 min (UTMD approach). The fluorescent image was captured using Xenogen IVIS spectrum. Release and tumor specific delivery of labeled His-MDA-7 was mostly localized in the left flank where ultrasound was applied. Lighter color indicates higher fluorescent intensity.

FIG. 4 shows covalently attached cVEGF-decorated MBs adhere to murine MC38 tumor vasculature, following blood clearance of circulating bubbles (left panel). Sequoia Cadence CPS imaging mode. MB s decorated with anti-VCAM-1 antibody (nanobody) fragment (right panel, top frame) or a control antibody fragment (bottom frame). Comparison relative to the control shows accumulation of the targeted MBs in the MC38 murine tumor vasculature.

FIGS. 5A-5D show site specific delivery of Ad.5/3-CMV-luc using targeted or decorated microbubble (D-MB) in immunocompetent prostate cancer Hi-myc and breast cancer MMTV-PyMT mice. Biotinylated anti-V-CAM-1 (B-VCAM-1) (100 μg) was incubated with Streptavidin microbubble (MB-SA) (˜10⁹ MB particles) that formed the complex Biotin-anti-V-CAM-1-Streptavidin-MB (MB-SA-B-anti-VCAM-1; D-MB). In-order to validate the preparation of D-MB, both the D-MB as well as simple MB-SA was mixed with Avidin-FITC, and flow-cytometry was done to confirm the formation of D-MBs (FIG. 5A). D-MB and simple MBs complexed with Ad.5/3-CMV-luc were systemically injected via the tail vein and sonoporated as indicated by the dashed circle in Hi-myc (FIG. 5B) and MMTV-PyMT (FIG. 5C) using FUS after 6 min of post injection of MB/Ad.5/3-CMV-luc. Bioluminescence imaging (BLI) was done after 72-h of post injection of D-MB/Ad.5/3-CMV-luc using IVIS spectrum. BLI image of dissected tumor and organs of MMTV-PyMT mice injected with D-MB/ad.luc followed by ultrasound targeted MB destruction (UTMD) at the site of tumor (FIG. 5D).

FIGS. 6A and 6B show specific delivery of adenovirus (Ad) by targeted MBs (anti PSMA-MBs), according to an embodiment. FIG. 6A shows tumor xenografts were developed after subcutaneous (s.c.) injection of PC-3 on the left flank and PC-3-PIP (PC-3 overexpressing PSMA) on the right flank. After tumor formation, Ad.5/3-CMV-luc alone, or complexed with plain (undecorated) or targeted (decorated) MBs (anti-PSMA-MBs) were injected via tail vein injection and after 10 min post-injection, mice were sonoporated on the right flank by using ultrasound transducer for 10 min. FIG. 6B shows image acquisition after 72-h following sonoporation using an IVIS system, and the image was analyzed by Living Image 4.3.1. It showed clearly that targeted (decorated) MBs provided more specific delivery of Ads without any non-specific delivery in adjacent organs, whereas plain (undecorated) MBs also delivered Ads to the adjacent tissues or organs (liver, spleen) in addition to tumor site where ultrasound was applied.

FIGS. 7A-7D show Focused Ultrasound (FUS) Dual MB (FUS-DMB) delivery of Ad.5/3-CTV significantly prolongs the survival of human GBM tumor bearing mice. FIG. 7A, schematic diagram showing FUS-DMB-Ad.5/3-CTV administration. FIG. 7B, luciferase expressing primary human GBM6 tumors were established in nude mice. The mice were treated with intravenous injections of either Ad.5/3-null with FUS-DMB (left panel), Ad.5/3-CTV with DMB (No FUS/center panel) or Ad.5/3-CTV with FUS-DMB (right panel). Representative BLI images are shown. FIG. 7C, Kaplan Meier analysis showing percent survival of GBM6 implanted mice treated as in B. *p<0.001 vs. control/DMB-CTV (No FUS). FIG. 7D, mice were euthanized when they reach IACUC end points and brains were collected and fixed in Formalin. FFPE tissue sections were stained for MDA-7/IL-24 (transgene expression), Ki-67 (proliferation marker), CD-31 (angiogenesis marker) and GRP-78 (established downstream target of MDA-7/IL-24). Only FUS-DMB CTV treatment enhanced MDA-7 and GRP-78 expression and decreased Ki-67 and CD31 expression, as expected.

FIGS. 8A and 8B show FUS-DMB or direct intracranial delivery of Ad.5/3-CTV significantly and comparably prolongs the survival of glioma stem cell (GSC) tumor-bearing mice. Luciferase expressing GSC-8-11 tumors were established in nude mice. FIG. 8A, mice were either treated with direct intracranial injection of Ad.5/3-CTV or intravenous injections of Ad.5/3-CTV with FUS-DMB and mice were observed using IVIS imaging. Representative images of BLI are shown. FIG. 8B, Kaplan Meier analysis showing survival analysis of GSC-8-11 implanted mice with FUS-DMB-Ad.5/3-CTV or IC-Ad.5/3-CTV treatment. In both treatment protocols, significant prolonged survival gains were observed. These observations are intriguing and underscore the power of our FUS-DMB approach as a novel treatment option to treat GBM patient in a non-surgical and non-invasive manner. *p<0.001 vs. control IC; @p<0.001 vs. control.

FIGS. 9A and 9B show multiple injections with Ad.5/3-CTV extend further the survival of GSC-driven tumor-bearing mice. FIG. 9A, mice were either treated with a single intravenous injection of Ad.5/3-CTV with FUS-DMB or multiple injections of Ad.5/3-CTV with FUS-DMB and mice were observed using IVIS imaging. Representative images of BLI are shown. FIG. 9B,

Kaplan Meier analysis showing survival analysis of GSC-8-11 implanted mice treated as in A. This study establishes that Ad.5/3-CTV with FUS-DMB can be administered multiple times without any toxic effect to in animals and multiple administrations of Ad.5/3-CTV with FUS-DMB enhance further the survival of glioma-bearing mice than with a single administration. The FUS-DMB approach involves non-invasive intravenous administration of Ad.5/3-CTV without surgery. *p<0.001 vs. control.

FIG. 10 shows FUS-DMB approach can specifically target a “theranostic” TCTV virus to the brain and can non-invasively image GBM in mice brain. GBM6 Cells were injected intracranially and treated with FUS-DMB containing Ad.5-TCTV. Mice were imaged 24 hours after treatment using an IVIS imager. Representative BLI images are shown. Left panel, Mice were injected intravenously with DMB-Ad.5-TCTV but no FUS was applied. Right panel, Mice were injected intravenously with DMB-Ad.5-TCTV and FUS was applied in the brain region (FUS-DMB approach). TCTV is a unique tripartite theranostic virus that employs three distinct promoters to target virus replication, cytokine production and imaging capabilities uniquely in cancer cells. Conditional replication of the TCTV is regulated by a cancer-selective (truncated PEG-3) promoter, the therapeutic component, MDA-7/IL-24, is under a ubiquitous (CMV) promoter, and finally the imaging capabilities are synchronized through another cancer selective (truncated tCCN1) promoter. This study suggests that FUS-DMB-TCTV can noninvasively image and treat glioma in mice brain.

FIG. 11 shows schematic diagram showing FUS-DMB-approach in the pancreas. The FUS-DMB approach can be used to deliver viruses systemically in the pancreas, and theoretically other organ sites in the body using focused ultrasound. The FUS-DMB approach is more effective in delivery of viruses than using a single FUS MB approach (UTMD-ultrasound targeted microbubble destruction approach).

FIGS. 12A and 12B show systemic administration of Ads using FUS-DMB to target the pancreas. KPC (Pdx-1-Cre/K-ras^{LSL-G12D}/p53^{fl/fl}) homozygous mice were injected with Ad.5/3-Luc/MB complex through the tail vein and the MBs were allowed to circulate for 15 sec. FUS was applied to the pancreas region with an immersion transducer using 10 dB amplitude, 1 MHz frequency and 3.5 mV power for 1 min. Mice were imaged using Xenogen IVIS spectrum imager 48 hr after transduction. The indicated organs were collected (Li-Liver, Lu-Lung, P-Pancreas, Sp-Spleen) and ex vivo imaging was performed with BLI. FIG. 12A, only I.V. (intravenous) injection of Ad.5/3-Luc, no FUS resulted in accumulation of luciferase signals mostly in the liver. FIG. 12B, I.V. (intravenous) injection of Ad.5/3-Luc, with FUS-DMB in the pancreas region results in accumulation of luciferase mostly in the pancreas.

FIGS. 13A and 13B show systemic administration of Ads expressing shRNAs using FUS-DMB in the pancreas specifically decrease target gene expression in the pancreas. MBs/Ads (Ad.shmda-9) were systemically injected via tail vein (100 μl) in KPC mice using FUS-DMB. Mice were maintained for 48 hours, euthanized and organs (spleen, pancreas, lungs and liver) were collected. These organs were lysed and RNA/protein was isolated using standard protocols. FIG. 13A, an equal amount of RNA was used to synthesize cDNA and real-time PCR was performed to check MDA-9/Syntenin/SDCBP mRNA levels. Mouse GAPDH was used as a transcription control. *p<0.05 vs. control. FIG. 13B, western blotting analysis of MDA-9/Syntenin/SDCBP. β-Actin was used as a loading control. This study establishes that Ad.shMDA-9 with FUS-DMB can be administered intravenously and can inhibit MDA-9/Syntenin/SDCBP levels specifically in the pancreas as compared to other organs. It also shows that Ad.shMDA-9 with FUS-DMB can be used to target the pancreas that is not evident in untreated mice (not receiving FUS).

FIGS. 14A and 14B show dual MB approach is more efficient that a single MB approach. MB s/Ads (Ad.shmda-9) were systemically injected via tail vein (100 μl) in KPC mice using either single MB or FUS-DMB. Mice were euthanized and organs (spleen, pancreas, lungs and liver) were collected at the indicated time points. These organs were lysed, and RNA/protein was isolated using standard protocols. FIG. 14A, an equal amount of RNA was used to synthesize cDNA and real-time PCR was performed to check MDA-9/Syntenin/SDCBP mRNA levels. Mouse GAPDH was used as a transcription control. *p<0.05 vs. control. FIG. 14B, western blotting analysis of MDA-9/Syntenin/SDCBP. β-Actin was used as a loading control. This study establishes that Ad.shMDA-9 with FUS-DMB can be administered and it is more efficient in inhibiting MDA-9/Syntenin/SDCBP levels in the pancreas as compared to a FUS-MB (single microbubble delivery).

FIG. 15 shows FUS-DMB delivery of Ad.5/3-shMDA-9 significantly prolongs the survival of pancreatic tumor-bearing mice. First, we injected empty microbubbles and applied FUS for 1 minute using same parameters described in FIGS. 12A and 12B and a minute later, complement-treated MBs/Ad.shMDA-9 were systemically injected via tail vein (100 μl ) in KPC homozygous mice and sonoporated using FUS. These mice then received intraperitoneal injection of Gemcitabine (20 mg/Kg) after 24 and 48 hours of Ad.shMDA-9 injection. The same process was repeated twice (total injection of Ad.shMDA-9 +FUS-DMB: 3; Gemcitabine: 6). These mice were observed for survival and Kaplan Meier analysis was performed. *p<0.01 vs. control; **p<0.001 vs. control. This study establishes that Ad.shMDA-9 with FUS-DMB can be administered multiple times without known toxicity in mice and can be combined with chemotherapeutic agents for better therapeutic effects.

### DETAILED DESCRIPTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

Section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entireties for any purpose.

### Definitions

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); and Sambrook and Green, Molecular Cloning: A Laboratory Manual, 4th Edition (2012). Methods, devices and materials similar or equivalent to those described herein can be used in the practice of this invention. The following definitions are provided to facilitate understanding of certain terms used frequently herein and are not meant to limit the scope of the present disclosure.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

It is noted that, as used herein and in the appended claims, the singular forms “a,” “an,” and “the” include plural referents unless the context clearly dictates otherwise. It is further noted that the claims may be drafted to exclude any optional element. As such, this statement is intended to serve as support for the recitation in the claims of such exclusive terminology as “solely,” “only,” and the like in connection with the recitation of claim elements, or use of a “negative” limitations, such as “wherein [a particular feature or element] is absent,” or “except for [a particular feature or element],” or “wherein [a particular feature or element] is not present (included, etc.) . . . .”

The terms “nucleic acid,” “nucleic acid molecule,” “nucleic acid oligomer,” “oligonucleotide,” “nucleic acid sequence,” “nucleic acid fragment,” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics, which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, of any length. The polymer may be linear or branched, it may comprise modified amino acids, and it may be interrupted by non amino acids. The terms also encompass an amino acid polymer that has been modified; for example, by disulfide bond formation, glycosylation, lipidation, acetylation, phosphorylation, or any other manipulation, such as conjugation with a labeling component. A “fusion protein” refers to a chimeric protein including two or more separate protein sequences that are recombinantly expressed as a single moiety.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a sequence comparison algorithm (optionally, with default parameters) or by manual alignment and visual inspection. In embodiments, sequences that are “substantially identical” are at least 80%, 90%, 95%, 99%, or more identical. In the case of nucleic acids, percent identity may also refer to, or may be applied to, the complement of a test sequence. As described below, the preferred algorithms can account for gaps and the like. In embodiments, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions as compared to the reference sequence (which does not comprise the additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison (e.g., with respect to the reference sequence), and multiplying the result by 100 to yield the percentage of sequence identity. Programs for determining sequence identity are known to those skilled in the art, and include, without limitation, BLAST (see, e.g., NCBI web site www.ncbi.nlm.nih.gov/BLAST or the like, optionally using default parameters), the Needleman-Wunsch algorithm (see e.g. the EMBOSS Needle aligner available at www.ebi.ac.uk/Tools/psa/emboss_needle/, optionally with default settings).

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence. Amino acid mutations may be identified by a designation identifying the original amino acid (e.g., as in a wild-type or reference sequence), the position of the mutation, and the amino acid to which the original amino acid was changed. For example, “K122R relative to SEQ ID NO: 2” indicates a mutation of the lysine at position 122 of SEQ ID NO: 2 to an arginine. Nucleotide mutations can use a similar designation scheme.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. Whether an amino acid corresponds to a particular position in a reference sequence (e.g., a mutation of K122R relative to SEQ ID NO: 2), optionally at a different position, can be determined by sequence alignment. In general, an alignment showing identity of one or more amino acids flanking the indicated position of the reference sequence will allow the corresponding position of the query sequence to be positioned locally with respect to the reference sequence to confirm the presence of a mutation of the corresponding amino acid, optionally at a shifted numerical position in the query sequence. In embodiments, a region comprising at least three to fifteen amino acids, including the mutation position, will locally align with the corresponding reference sequence with a relatively high percent identity, except for the position of the mutant amino acid along the query sequence (e.g. at least about 90%, 95%, or 100% identity). In embodiments, an amino acid of a query MDA-7/IL-24 protein sequence corresponds to a particular position of a reference sequence if the polypeptide of the query sequence aligns to the particular position of the reference sequence when the two sequences are optimally aligned using a BLASTP alignment algorithm with default parameters.

The terms “MDA-7”, “IL-24”, or “MDA-7/IL-24” refer to a protein (including homologs, isoforms, and functional fragments thereof) with MDA-7 activity. The term includes any recombinant or naturally-occurring form of MDA-7 or variants, homologs, or isoforms thereof that maintain MDA-7 activity (e.g. within at least 30%, 40%, 50%, 60%, 70%, 80%, 90%, 95%, or 100% activity compared to wild-type MDA-7). In embodiments, the variants, homologs, or isoforms have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring MDA-7 protein. In embodiments, the MDA-7 protein is substantially identical to the protein identified by Accession No. NP_006841 or a variant or homolog having substantial identity thereto. In embodiments, the MDA-7 protein is substantially identical to the protein identified by UniProt Q13007 or a variant or homolog having substantial identity thereto. In embodiments, the IL-24 gene is substantially identical to the nucleic acid sequence set forth in RefSeq (mRNA) NM_006850, or a variant or homolog having substantial identity thereto. In embodiments, the IL-24 gene is substantially identical to the nucleic acid sequence set forth in Ensembl reference number ENSG00000162892, or a variant or homolog having substantial identity thereto. In embodiments, the amino acid sequence or nucleic acid sequence is the sequence known at the time of filing of the present application. In embodiments, the protein is a precursor form that includes a signal sequence. In embodiments, the signal sequence is not the native MDA-7 signal sequence, such as a modified native signal sequence, an unmodified signal sequence from another gene (e.g., the insulin gene), or a modified signal sequence from another gene. In embodiments, the protein is a mature form of MDA-7, in which a signal sequence at the N-terminus of a precursor form of the protein is absent. The mature form can be produced post-translationally from a precursor form containing a signal sequence, or can be translated directly from a polynucleotide encoding the mature form without a signal sequence N-terminal with respect to the sequence of the mature MDA-7. In embodiments, the MDA-7/IL-24 protein comprises SEQ ID NO: 4, or variants, homologs, or isoforms thereof that maintain or enhance MDA-7 activity. In embodiments, the MDA-7/IL-24 protein comprises SEQ ID NO: 3, or variants, homologs, or isoforms thereof that maintain or enhance MDA-7 activity. In embodiments, the MDA-7/IL-24 protein does not comprise the first 49 amino acids of SEQ ID NO: 2. In embodiments, the MDA-7/IL-24 protein comprises SEQ ID NO: 18, or variants, homologs, or isoforms thereof that maintain or enhance MDA-7 activity. Additional non-limiting examples of MDA-7 polynucleotide and polypeptide sequences are described in US20200354745A1, which is incorporated herein by reference.

The terms “MDA-9”, “Syntenin”, “Syndecin Binding Protein”, “SDCBP” or “MDA-9/Syntenin” refer to a protein (including homologs, isoforms, and functional fragments thereof) with MDA-9 activity. The term includes any recombinant or naturally-occurring form of MDA-9 or variants, homologs, or isoforms thereof that maintain MDA-9 activity (e.g. within at least 30%, 40%, 50%, 60%, 70%, 80%, 90%, 95%, or 100% activity compared to wild-type MDA-9). In embodiments, the variants, homologs, or isoforms have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring MDA-9 protein. In embodiments, the MDA-9 protein is substantially identical to the protein identified by Accession No. NP_005616 or a variant or homolog having substantial identity thereto. In embodiments, the MDA-9 protein is substantially identical to the protein identified by UniProt 000560 or a variant or homolog having substantial identity thereto. In embodiments, the MDA-9 gene is substantially identical to the nucleic acid sequence set forth in RefSeq (mRNA) NM_005625, or a variant or homolog having substantial identity thereto. In embodiments, the SDCBP gene is substantially identical to the nucleic acid sequence set forth in Ensembl reference number ENSG00000137575, or a variant or homolog having substantial identity thereto. In embodiments, the amino acid sequence or nucleic acid sequence is the sequence known at the time of filing of the present application. In embodiments, the protein is a precursor form that includes a signal sequence. Additional non-limiting examples of MDA-9 polynucleotide and polypeptide sequences are described in WO2017120439, which is incorporated herein by reference.

The terms “signal sequence” and “signal peptide” refer to a polypeptide sequence that is capable of directing the secretion of a protein that includes the signal peptide. Typically, a signal peptide is at or near the N-terminus of a protein. The signal peptide may be immediately adjacent to the protein to be secreted, or may be joined by a linker of one or more amino acids. In eukaryotes, secretion typically involves directing a protein to the endoplasmic reticulum, and may involve cleavage to remove some or all of the signal peptide prior to secretion out of the cell. In bacteria, proteins may be secreted to the periplasm or into the medium. A signal peptide is capable of directing the secretion of a protein that includes the signal peptide if, when the signal peptide is attached to a protein of interest (e.g., an MDA-7/IL-24 protein), more of the protein of interest is secreted from a cell than in the absence of the signal peptide. In embodiments, at least about 10%, 20%, 30%, 40%, 50%, 60%, 70%, 80%, 90%, 95% or more of the protein of interest is secreted. In embodiments, at least 50% of the protein of interest is secreted. Secretion can be measured in any suitable system, such as in cultured cells described herein. In embodiments, the signal sequence is joined to a protein of interest such that cleavage during the secretion process removes the entire signal sequence.

One of skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

Certain amino acids may be substituted for other amino acids in a protein structure without appreciable loss of tumoricidal effects. Since it is the interactive capacity and nature of a protein that defines that protein's biological functional activity, certain amino acid substitutions can be made in a protein sequence, and, of course, in its DNA encoding sequence, and nevertheless obtain a protein with like properties. It is thus contemplated that various changes may be made in the polypeptide sequences of the present disclosure, or corresponding DNA sequences which encode said polypeptides, while retaining at least some of their biological activity. Such biological activity can be assessed by various techniques, such as for instance assays described in the examples herein.

The term “purified,” when applied to a nucleic acid or protein, denotes that the nucleic acid or protein is essentially free of one or more other cellular components with which it is associated in the natural state or in a whole cell lysate. It can be, for example, in a homogeneous state or in a mixture with one or more other compounds, and may be in either a dry or aqueous solution. For example, an MDA-7/IL-24 protein (or a polynucleotide or vector encoding the same) may be purified from a cell lysate, then combined with one or more other agents (e.g., microbubbles, and optionally an anticancer agent). As such, compositions comprising a purified MDA-7/IL-24 protein (or a polynucleotide or vector encoding the same) may comprise additional compounds, but will generally lack or be reduced in one or more impurities present in a lysate or media from which an MDA-7/IL-24 protein (or a polynucleotide or vector encoding the same) is initially isolated. Purity and homogeneity are typically determined using analytical chemistry techniques such as polyacrylamide gel electrophoresis or high performance liquid chromatography. A molecule that is the predominant species present in a preparation is substantially purified.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head and neck, liver, kidney, lung, ovary, pancreas, rectum, stomach, and uterus. Additional examples include, thyroid carcinoma, cholangiocarcinoma, pancreatic adenocarcinoma, pancreatic ductal adenocarcinoma (PDAC), skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. In embodiments, the cancer is a cancer that metastasized to bone. In embodiments, the cancer is prostate cancer, such as prostate cancer-derived bone metastasis.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., prostate, which site is referred to as a primary tumor, e.g., primary prostate cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if prostate cancer metastasizes to the bone, the secondary tumor at the site of the bone consists of abnormal prostate cells and not abnormal bone cells. The secondary tumor in the bone is referred to as a metastatic bone cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the bone.

As used herein, a “subject” can be a mammal such as a non-primate (e.g., cows, pigs, horses, cats, dogs, rats, etc.) or a primate (e.g., monkey and human). In embodiments, the subject is a human. In embodiments, the subject is a mammal (e.g., a human) having or potentially having a cancer, such as a metastatic cancer, described herein. In embodiments, the subject is a mammal (e.g., a human) at risk of developing a cancer, such as a metastatic cancer, described herein.

“Treating” or “treatment” as used herein broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, delay or slowing of disease progression, amelioration or palliation of the disease state, diminishment of the reoccurrence of disease, and remission, whether partial or total and whether detectable or undetectable. In aspects, the subject has been previously treated for the disease. In other words, “treatment” as used herein includes any cure or amelioration of a disease. Treatment may relieve the disease's symptoms fully or partially remove the disease's underlying cause, shorten a disease's duration, or do a combination of these things. In the case of cancer, treatment may include slowing, halting, or reversing cancer cell multiplication (e.g., as in growth of a tumor, as measured by tumor size or a rate of change thereof).

“Preventing” as used herein refers to a decrease in the occurrence or incidence of one or more disease symptoms in a patient. Prevention may be complete (no detectable symptoms) or partial, such that fewer symptoms are observed than would likely occur absent treatment. Prevention includes prophylactic treatment.

The length of treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prevention may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, administering a composition of the present disclosure both treats a cancer of a subject (e.g., metastatic bone cancer), and prevents further disease systems (e.g., metastasis, such as bone metastases).

The compositions described herein can be used in combination with one another, or with other active agents known to be useful in treating a cancer, such as anti-cancer agents. “Anti-cancer agent” is used in accordance with its plain ordinary meaning and refers to a composition (e.g. compound, drug, antagonist, inhibitor, modulator) having antineoplastic properties or the ability to inhibit the growth or proliferation of cancer cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

As used herein, the term “administering” encompasses oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). In embodiments, “administering” a protein or a composition comprising the protein refers to administering the protein itself (e.g., an MDA-7/IL-24 protein), rather than a polynucleotide encoding the protein.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

Therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic composition sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side effects. Determination of the proper dosage for a particular situation is within the skill of the practitioner. Generally, treatment is initiated with smaller dosages that are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

As used herein, the term “microbubble” generally refers to any spherical arrangement of lipids creating an outer shell and an inner void space. The lipid layer may be modified to bind molecules in a stable manner, such as by incorporating an active agent as part of the outer shell while forming the microbubbles, or complexing the active agent with the shell after formation of the microbubbles (e.g., via non-covalent interaction). In embodiments, the use of microbubbles as vectors for delivery of active agents utilizes destruction of agent-loaded microbubbles by a focused ultrasound beam during their microvascular transit through the target area, resulting in localized transduction upon disruption of the microbubble shell, while sparing non-targeted areas (see, e.g., U.S. Patent App. Pub. No. 2013204166). Ultrasound/Microbubble Targeted Delivery (UMTD) has been used to deliver genes to cells in vitro, and more recently, has been employed to deliver genes in vivo to treat diabetes and cardiovascular disease in experimental animal models (Chen et al. (2007) Gene Ther. 14:1102-1110; Fujii et al. (2009) J. Am. Coll. Cardiol. Cardiovasc. Imaging 2:869-879). In some embodiments, the microbubbles are gene or molecular therapy vectors. The use of microbubbles as gene vectors has advantages over viral systems. During UMTD, intravenously injected microbubbles can be destroyed as they transit through the microcirculation of the target site where the ultrasound beam is directed, functionally achieving selective payload delivery without the need for invasive approaches such as direct intratumor injection. In embodiments, lipid microbubbles we used for UMTD are administered repetitively. In embodiments, because the microbubbles are ultrasound contrast agents, it is possible to simultaneously image microbubble transit through a target tissue (e.g., a tumor), thereby enabling more precise real time guidance of active agent delivery. Any of a variety of procedures may be used in the formation of microbubbles from a variety of suitable materials. For example, commercial available compositions useful for forming microbubbles include, without limitation, SONAZOID, OPTISON, SONOVUE, MICROMARKER, POLYSON, and other such ultrasound imaging agents. Microbubbles may be formed from lipids including, but not limited to, dipalmitoyl and distearoyl phosphatidic acid (DPPA, DSPA), dipalmitoyl and distearoyl phosphatidylserine (DPPS, DSPS), phosphatidyl glycerols such as dipalmitoyl and distearoyl phosphatidylglycerol (DPPG, DSPG), 1,2-bis(10,12-tricosadiynoyl-sn-glycero-3-phosphocobne, L-a-phosphatidylcholine, PE-PEG2000 (1,2-distearoyl-sn-glycero-3-phosphoethanolamine-N-[methoxy(polyethylene glycol)-2000, PE-PEG2000-biotin, and combinations of one or more of these. Non-lipids (e.g., proteins, and/or one or more active agents) may be included to form part of the outer shell of a microbubble in complex with one or more lipids of the shell. The inner void space may be occupied by a suitable gas, such as air or perfluorobutane. Additional non-limiting examples of gases are described in US20160243234A1, which is incorporated herein by reference. In general, microbubbles have an average or median diameter of about 0.1 microns to about 100 micros. Further non-limiting examples of compositions for the formation of microbubbles are described in US20160108429A1 and WO2020118271A1, which are incorporated herein by reference.

As used herein, the term “target tissue” refers to any ensemble of related or similar cells. Non-limiting examples of target tissue include connective, muscle, nervous, or epithelial. Target tissue may include epithelial tissue that forms the surfaces of the skin, airways, soft organs, reproductive tract, the inner lining of the digestive tract; fibrous connective tissue, skeletal connective tissue, fluid connective tissue, vasculature, bone, ligament, tendon, blood, blood vessels, adipose, areolar, skeletal muscle, smooth muscle, cardiac muscle, neural tissue of the brain, neural. tissue of the brain, neural tissue of the spinal cord, neural tissue of the cranial neurons, neural tissue of the spinal neurons. The target tissue may be healthy or diseased (e.g. cancerous). The target tissue may be derived from a living organism or grown in vitro. The target tissue may be transplanted.

As used herein, the term “theranostic” refers to a combination of the terms therapeutic and diagnostic. A non-limiting example of a theranostic composition is a composition that can both be used to image and treat a target tumor in a subject.

As used herein, the term “active agent” refers to a compound that is a therapeutic agent, an imaging agent, or an theranostic agent.

As used herein, the term “microbubble-enclosed active agent” refers to a compound that is a therapeutic agent, an imaging agent, or an agent that is both a therapeutic and an imaging agent and is also enclosed within a microbubble.

As used herein, the term “microbubble-excluded active agent” refers to a compound that is a therapeutic agent, an imaging agent, or an agent that is both a therapeutic and an imaging agent and is also excluded from a microbubble.

As used herein, a “therapy that comprises microbubbles” refers to any therapy that comprises treatment with one or more active agents and also comprises a microbubble.

As used herein, a “therapy that does not comprise microbubbles” refers to any therapy that comprises treatment with one or more active agents, without comprising a microbubble.

A “therapeutic agent” as used herein refers to an agent (e.g., compound or composition described herein) that when administered to a subject will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being. In embodiments, the therapeutic agent is an anticancer agent. In embodiments, the therapeutic agent is a nucleic acid, a protein, or a vector (e.g., a plasmid or a virus).

“Anti-cancer agent” and “anticancer agent” are used in accordance with their plain ordinary meaning and refers to a composition (e.g. compound, drug, antagonist, inhibitor, modulator) having antineoplastic properties or the ability to inhibit the growth or proliferation of cells. In some embodiments, an anti-cancer agent is an alkylating agent, an antimetabolite, a natural product, or a hormone. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/ AZD6244, GSK1120212/ trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5- azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HC1, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (-)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

An “imaging agent” is a compound that allows for the detection, imaging, and/or monitoring of the presence and/or progression of a condition, pathological disorder, and/or disease. Imaging agents include compounds that are detectable by spectroscopic, photochemical, biochemical, immunochemical, chemical, or other physical means. Exemplary imaging agents include, without limitation, ³²P radionuclides, positron-emitting isotopes, fluorescent dyes, fluorophores, antibodies, bioluminescent molecules, chemiluminescent molecules, photoactive molecules, metals, electron-dense reagents, enzymes (e.g., as used in an ELISA), magnetic contrast agents, quantum dots, nanoparticles (e.g. gold nanoparticles), biotin, digoxigenin, haptens and proteins or other entities which can be made detectable, e.g., by incorporating a radiolabel into a peptide or antibody specifically reactive with a target peptide. Any method known in the art for conjugating an antibody to a label may be employed. Exemplary fluorophores include fluorescein, rhodamine, GFP, coumarin, FITC, Alexa fluor®, Cy3, Cy5, BODIPY, and cyanine dyes. Exemplary radionuclides include Fluorine-18, Gallium-68, and Copper-64. Exemplary magnetic contrast agents include gadolinium, iron oxide and iron platinum, and manganese. In some embodiments, the imaging moiety is a bioluminescent molecule.

As used herein, the term “targeting moiety” and its equivalents refer to a molecule that recognizes and binds to a desired molecule or structure on the surface of a cell or tissue, such that it directs complexes with which it is associated to preferentially accumulate at a target site, relative to non-target sites. Non-limiting examples of targeting moieties include antibodies, antibody fragments, binding proteins and peptides, receptors and ligands for receptors. A specific binding partner for a targeting moiety means that the targeting moiety binds with greater specificity to the target molecule or structure than it does to non-target molecules or structures (e.g., at least 2-fold, 5-fold, 10-fold, 100-fold, or higher specificity). In embodiments, the targeting moiety is a member of a known binding pair (e.g., antibody/antigen, ligand/receptor, and lectin/carbohydrate). In embodiments, complexes comprising a targeting moiety accumulate at or in a target tissue that is distal to a site of administration to a greater degree than comparable complexes lacking the targeting moiety.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv , dsFv or Fab; while the terms “variable light chain,” “V_{L}” or “VL” refer to the variable region of an immunoglobulin light chain, including of an Fv, scFv , dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992)Biochemistry 31:1579, Hollinger et al.(1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

The term “virus” refers to a submicroscopic infectious agent that only replicates within a host cell. The genetic material of a virus can be either DNA or RNA.

The term “adenovirus” or “Ad” refers to a virus of the family Adenoviridae. Adenoviruses are non-enveloped, icosahedral shaped, medium sized (90-100 nm diameter), double stranded DNA viruses that are found in a large range of vertebrate hosts.

The term “viral tropism” refers to the ability of a virus to infect a specific cell type and ultimately produce a successful infection.

The term “tropism-modified AdV” refers to an adenovirus that has been genetically engineered to have an alternative tropism from its innate tropism. For example, the tropism of HAd5 is predominantly mediated by the interaction of fiber/knob with primary adenovirus receptor, the coxsackie and adenovirus receptor (CAR) CAR. To bypass the dependence on CAR for adenoviral entry and replication, a number of different approaches have been used in the past few years. For example, several groups have genetically modified the knob domain of fiber in an attempt to retarget Ad vectors. Genetic alterations include virions containing chimeric fiber proteins composed of the tail and shaft domains of adenovirus type 5 (Ad5) fiber and the knob domain of Ad3 or the exchange of fiber with alternative serotypes such as Ad11 and Ad35. Ad3 virus can bind to Desmoglein and CD46, so these viruses with Ad.5/3 chimeric structure can bind CAR, Desmoglein and CD46, increasing their ability to infect cells with reduction in any of these receptors. A different approach includes the incorporation of COOH-terminal polylysine sequences or an integrin-binding RGD motif at the COOH terminus of Ad5 fiber. Examples of tropism-modified adenoviruses include, but are not limited to, Ad.5/3-CTV, Ad5/3-C-RGD, Ad5/3-HI-RGD, Ad5/3-E2F-d24, Ad5.RGD.pK7.

The term “CTV” or “cancer terminator virus” refers to a virus of the family Adenoviridae that has been modified by modern microbiological engineering techniques. Non-limiting examples of cancer terminating viruses include a theranostie tripartite CR (TCTV) that selectively expresses three genes from three distinct promoters (e.g. Ad.5-TCTV, Ad.5/3-TCTV) as described in Bhoopathi, P., et al.. (2021) Cancers, 13(4), 85, incorporated herein by reference, and a tropism modified cancer terminator virus (e.g., A.d.5/3.-CTV; Ad.5/3.-CTV-M7) as described in WO2014093270A1, incorporated herein by reference.

### Compositions and Kits

In some aspects, the present disclosure provides compositions and kits for use in treatment of a target tissue with an active agent. In embodiments, the kit includes a first and second microbubble composition and an active agent. In some embodiments, the first microbubble composition does not include the active agent. In some embodiments, the second microbubble composition includes the active agent. In some embodiments, the active agent is a therapeutic agent. In some embodiments, the active agent is an imaging agent.

In embodiments, the first and second microbubble compositions are formulated for intravenous administration. In some embodiments, the first composition is formulated for intravenous administration. In some embodiments, the second composition is formulated for intravenous administration.

In embodiments, the first and/or second microbubbles have a mean or median diameter of about 1 micron to about 50 microns, about 1 micron to about 25 microns, about 1 micron to about 10 microns, or about 1 micron to about 5 microns. In embodiments, the first and/or second microbubbles have a mean or median diameter of about 1 micron to about 5 microns. In embodiments, the first and/or second microbubbles have a mean or medium diameter of about 2.5 microns to about 4 microns.

In some embodiments, the first microbubbles have a mean or median diameter of about 1 micron. In some embodiments, the first microbubbles have a mean or median diameter of about 2 microns. In some embodiments, the first microbubbles have a mean or median diameter of about 3 microns. In some embodiments, the first microbubbles have a mean or median diameter of about 4 microns. In some embodiments, the first microbubbles have a mean or median diameter of about 5 microns. In some embodiments, the first microbubbles have a mean or median diameter of about 1 micron to about 2 microns. In some embodiments, the first microbubbles have a mean or median diameter of about 1 micron to about 3 microns. In some embodiments, the first microbubbles have a mean or median diameter of about 1 micron to about 4 microns. In some embodiments, the first microbubbles have a mean or median diameter of about 2 microns to about 3 microns. In some embodiments, the first microbubbles have a mean or median diameter of about 2 microns to about 4 microns. In some embodiments, the first microbubbles have a mean or median diameter of about 3 micron to about 4 microns. In some embodiments, the first microbubbles have a mean or median diameter of about 3 microns to about 5 microns.

In some embodiments, the second microbubbles have a mean or median diameter of about 1 micron. In some embodiments, the second microbubbles have a mean or median diameter of about 2 microns. In some embodiments, the second microbubbles have a mean or median diameter of about 3 microns. In some embodiments, the second microbubbles have a mean or median diameter of about 4 microns. In some embodiments, the second microbubbles have a mean or median diameter of about 5 microns. In some embodiments, the second microbubbles have a mean or median diameter of about 1 micron to about 2 microns. In some embodiments, the second microbubbles have a mean or median diameter of about 1 micron to about 3 microns. In some embodiments, the second microbubbles have a mean or median diameter of about 1 micron to about 4 microns. In some embodiments, the second microbubbles have a mean or median diameter of about 2 microns to about 3 microns. In some embodiments, the second microbubbles have a mean or median diameter of about 2 microns to about 4 microns. In some embodiments, the second microbubbles have a mean or median diameter of about 3 micron to about 4 microns. In some embodiments, the second microbubbles have a mean or median diameter of about 3 microns to about 5 microns.

In embodiments, the first and/or second microbubbles comprise a targeting moiety. In some embodiments, the first microbubbles contain a targeting moiety. In some embodiments, the second microbubbles contain a targeting moiety. In some embodiments, the first and second microbubbles both contain a targeting moiety, which may be the same or different. In embodiments, the first and second microbubbles include a targeting moiety that binds the same target. In embodiments, the targeting moiety specifically binds a particular protein, a particular cell, or a particular tissue.

In embodiments, the microbubble comprises a targeting moiety. In embodiments, non-limiting examples of a targeting moiety comprise an antibody, an antibody fragment, a binding protein, a binding protein fragment, a receptor, a receptor fragment, a receptor ligand, a peptide, a polypeptide, a polynucleic acid, a polysaccharide, a lipid, a polymer, tumor-associated antigen, tissue specific antigen, a vascular associated antigen, or any combination of molecules thereof. In some embodiments, the targeting moiety is an antibody. In some embodiments, the targeting moiety is an antibody fragment. In some embodiments, the targeting moiety is a binding protein. In some embodiments, the targeting moiety is a binding protein fragment. In some embodiments, the targeting moiety is a receptor. In some embodiments, the targeting moiety is a receptor fragment. In some embodiments, the targeting moiety is a receptor ligand. In some embodiments, the targeting moiety is a peptide. In some embodiments, the targeting moiety is a polypeptide. In some embodiments, the targeting moiety is a polynucleic acid. In some embodiments, the targeting moiety is a polysaccharide. In some embodiments, the targeting moiety is a lipid. In some embodiments, the targeting moiety is a polymer. In some embodiments, the targeting moiety is a tumor-associated antigen. In some embodiments, the targeting moiety is a tissue specific antigen. In some embodiments, the targeting moiety is a vascular associated antigen. In some embodiments, the targeting moiety is any combination of the molecules described herein.

In embodiments, the tumor-associated antigen is selected from HER2, CEA, PSA, MUC1, PSMA, CA19-9, EpCAM, GPC3, mesothelin (MSLN), or EGFR. In embodiments, the tumor associated antigen is HER2. In embodiments, the tumor associated antigen is CEA. In embodiments, the tumor associated antigen is PSA. In embodiments, the tumor associated antigen is MUC1. In embodiments, the tumor associated antigen is PSMA. In embodiments, the tumor associated antigen is CA19-9. In embodiments, the tumor associated antigen is EpCAM. In embodiments, the tumor associated antigen is GPC3. In embodiments, the tumor associated antigen is mesothelin (MSLN). In embodiments, the tumor associated antigen is EGFR.

In embodiments, the tissue specific antigen is selected from Glycoprotein 2, Cadherin-9, GFAP, nestin, Tuj-1, Thymocyte antigen 1 (Thy1)/CD90, Desmin, Cx43. In embodiments, the tissue specific antigen is Glycoprotein 2. In embodiments, the tissue specific antigen is Cadherin-9. In embodiments, the tissue specific antigen is GFAP. In embodiments, the tissue specific antigen is nestin. In embodiments, the tissue specific antigen is Tuj-1. In embodiments, the tissue specific antigen is Thymocyte antigen 1 (Thy1)/CD90. In embodiments, the tissue specific antigen is Desmin. In embodiments, the tissue specific antigen is Cx43.

In embodiments, the targeting moiety comprises a VEGF polypeptide or single-chain variant thereof, which binds a VEGF receptor. In some embodiments, the targeting moiety is a VEGF polypeptide. In some embodiments, the targeting moiety is a single-chain variant of VEGF. Non-limiting examples of VEGF polypeptides and single-chain variants thereof useful as targeting moieties are provided in US20080312410A1, which is incorporated herein by reference.

In embodiments, the targeting moiety comprises a VCAM1 antibody or epitope-binding fragment thereof. In some embodiments, the targeting moiety is a VCAM1 antibody. In some embodiments, the targeting moiety is a VCAM1 epitope-binding fragment of an antibody.

In embodiments, the targeting moiety comprises a PSMA antibody or epitope-binding fragment thereof. In some embodiments, the targeting moiety is a PSMA antibody. In some embodiments, the targeting moiety is a PSMA epitope-binding fragment of an antibody.

In embodiments, the active agent is an anti-cancer agent. Several suitable anti-cancer agents are available, non-limiting examples of which are provided herein. In embodiments, the active agent comprises a vector, such as a plasmid or a virus. Non-limiting examples of viruses are an adenovirus, a cancer terminator virus (CTV), a lentivirus, a retrovirus, a herpesvirus, a vaccinia virus, a genetically modified HIV, or a vesicular stomatitis virus. In some embodiments, the virus is an cancer terminator virus (CTV). In some embodiments, the virus is a lentivirus. In some embodiments, the virus is a retrovirus. In some embodiments, the virus is a herpesvirus. In some embodiments, the virus is a vaccinia virus. In some embodiments, the virus is a genetically modified human immune deficiency virus (HIV). In some embodiments, the virus is a vesicular stomatitis virus. In some embodiments, the virus is an adenovirus. In some embodiments, the replication of the virus is under control of a cancer-selective promoter.

In embodiments, the active agent is a theranostic virus. In embodiments, the theranostic virus is an adenovirus. In embodiments, the adenovirus is genetically engineered. In embodiments, the adenovirus is a tropism-modified virus. In embodiments, the adenovirus comprises a tissue-selective promoter. In embodiments, the adenovirus comprises a cancer-selective promoter. In embodiments, the adenovirus comprises a tissue-selective terminator. In embodiments, the adenovirus comprises a cancer-selective terminator. In some embodiments, the adenovirus comprises more than one cancer-selective promoter. In some embodiments, the adenovirus comprises more than one tissue-selective promoter. In some embodiments, the adenovirus comprises more than one cancer-selective terminator. In some embodiments, the adenovirus comprises more than one tissue-selective terminator. In some embodiments, the adenovirus is a tropism modified cancer terminator virus.

In embodiments, the active agent comprises a protein or nucleic acid. In some embodiments, the active agent is a protein. In some embodiments, the active agent is a nucleic acid. In some embodiments, the active agent is a short hairpin RNA (shRNA). In some embodiments, the active agent is a small interfering RNA (siRNA). In some embodiments, the active agent is an antisense RNA. In some embodiments, the active agent in a lncRNA. In some embodiments, the active agent is DNA. In embodiments, the DNA encodes an shRNA or an antisense RNA.

In embodiments, the active agent comprises an MDA-9/Syntenin inhibitor. The MDA-9/Syntenin inhibitor can inhibit MDA-9/Syntenin activity at the polypeptide or polynucleotide level. In embodiments, the active agent comprises the nucleic acid sequence encoding part of the of MDA-9/Syntenin DNA sequence. In embodiments, the polynucleotide encodes a short hairpin RNA (shRNA) complementary to a portion of MDA-9/Syntenin sequence, a small interfering RNA (siRNA) complementary to a portion of MDA-9/Syntenin sequence, a microRNA (miRNA) complementary to a portion of MDA-9/Syntenin sequence, a messenger RNA (mRNA) complementary to a portion of MDA-9/Syntenin sequence, or an antisense RNA complementary to a portion of MDA-9/Syntenin sequence. In some embodiments, the polynucleotide encodes a short hairpin RNA (shRNA) complementary to a portion of MDA-9/Syntenin sequence. In some embodiments, the polynucleotide encodes a small interfering RNA (siRNA) complementary to a portion of MDA-9/Syntenin sequence. In embodiments, the polynucleotide encodes a microRNA (miRNA) complementary to a portion of MDA-9/Syntenin sequence. In embodiments, the polynucleotide encodes an antisense RNA complementary to a portion of MDA-9/Syntenin sequence. Compositions and methods MDA-9/Syntenin inhibitor are disclosed in International Patent Publications WO 2017/120439 and WO 2021/127305, which are herein incorporated by reference.

In embodiments, the active agent comprises the MDA-9/Syntenin polynucleotide sequence corresponding to the SEQ ID NO.: 19 and/or SEQ ID NO.: 20. In some embodiments, the active agent comprises the nucleic acid sequence SEQ ID NO.: 19. In some embodiments, the active agent comprises the nucleic acid sequence SEQ ID NO.: 20.

In embodiments, the active agent is a virus comprising a polynucleotide encoding part of the MDA-9/Syntenin nucleic acid sequence. In embodiments, the polynucleotide encodes a short hairpin RNA (shRNA) complementary to a portion of MDA-9/Syntenin sequence, a small interfering RNA (siRNA) complementary to a portion of MDA-9/Syntenin sequence, a microRNA (miRNA) complementary to a portion of MDA-9/Syntenin sequence, a messenger RNA (mRNA) complementary to a portion of MDA-9/Syntenin sequence, or an antisense RNA complementary to a portion of MDA-9/Syntenin sequence. In some embodiments, the polynucleotide encodes an short hairpin RNA (shRNA) complementary to a portion of MDA-9/Syntenin sequence. In some embodiments, the polynucleotide encodes a small interfering RNA (siRNA) complementary to a portion of MDA-9/Syntenin sequence. In embodiments, the polynucleotide encodes a microRNA (miRNA) complementary to a portion of MDA-9/Syntenin sequence. In embodiments, the polynucleotide encodes an antisense RNA complementary to a portion of MDA-9/Syntenin sequence.

In embodiments, the active agent comprises a virus containing the MDA-9/Syntenin polynucleotide sequence corresponding to the SEQ ID NO.: 19 and/or SEQ ID NO.: 20. In some embodiments, the active agent comprises the nucleic acid sequence SEQ ID NO.: 19. In some embodiments, the active agent comprises the nucleic acid sequence SEQ ID NO.: 20.

In embodiments, the active agent comprises a polynucleotide encoding an active RNA or protein, such as an MDA-7/IL-24 protein. In some embodiments, the active agent comprises a polynucleotide encoding an MDA-7/IL-24 fusion protein. In some embodiments, the active agent comprises a polynucleotide encoding an MDA-/IL-24 sequence variant.

In embodiments, the active agent is a virus containing a polynucleotide encoding an MDA-7/IL-24 protein. In some embodiments, the active agent is an adenovirus containing a polynucleotide encoding an MDA-7/IL-24 protein. In some embodiments, the active agent is a virus containing a polynucleotide encoding an MDA-7/IL-24 fusion protein. In some embodiments, the active agent is an adenovirus containing a polynucleotide encoding an MDA-7/IL-24 fusion protein.

In some embodiments, the active agent is a virus containing a polynucleotide encoding an MDA-7/IL-24 sequence variant. In some embodiments, the active agent is an adenovirus containing a polynucleotide encoding an MDA-7/IL-24 sequence variant.

In embodiments, the active agent is an MDA-7/IL-24 protein. In some embodiments, the MDA-7/IL-24 protein is a fusion protein. In some embodiments, the MDA-7/IL-24 protein is a sequence variant. In embodiments, the MDA-7/IL-24 protein comprises an insulin signal peptide.

In embodiments, the MDA-7/IL-24 protein includes an amino acid sequence of SEQ ID NO: 4, or a variant thereof. In embodiments, the MDA-7/IL-24 protein includes an amino acid sequence that is about or at least about 80%, 85%, 90%, 95%, 96%, 97%, 98%, 99%, or 100% identical to SEQ ID NO: 4. In embodiments, the MDA-7/IL-24 protein includes an amino acid sequence that is about or at least about 90% identical to SEQ ID NO: 4. In embodiments, the MDA-7/IL-24 protein includes an amino acid sequence that is about or at least about 95% identical to SEQ ID NO: 4.

In embodiments, the MDA-7/IL-24 protein includes an amino acid sequence that is about or at least about 80% identical to SEQ ID NO: 4. In embodiments, the MDA-7/IL-24 protein includes an amino acid sequence that is about or at least about 85% identical to SEQ ID NO: 4. In embodiments, the MDA-7/IL-24 protein includes an amino acid sequence that is about or at least about 90% identical to SEQ ID NO: 4. In embodiments, the MDA-7/IL-24 protein includes an amino acid sequence that is about or at least about 95% identical to SEQ ID NO: 4. In embodiments, the MDA-7/IL-24 protein includes an amino acid sequence that is about or at least about 96% identical to SEQ ID NO: 4. In embodiments, the MDA-7/IL-24 protein includes an amino acid sequence that is about or at least about 97% identical to SEQ ID NO: 4. In embodiments, the MDA-7/IL-24 protein includes an amino acid sequence that is about or at least about 98% identical to SEQ ID NO: 4. In embodiments, the MDA-7/IL-24 protein includes an amino acid sequence that is about or at least about 99% identical to SEQ ID NO: 4. In embodiments, the MDA-7/IL-24 protein includes an amino acid sequence that is about 100% identical to SEQ ID NO: 4. In embodiments, the MDA-7/IL-24 protein includes an amino acid sequence that is about or at least about 80%, 85%, 90%, 95%, 96%, 97%, 98%, 99%, or 100% identical across 25, 50, 75, or 100 continuous amino acids of SEQ ID NO: 4.

In embodiments, the MDA-7/IL-24 protein includes an amino acid sequence of SEQ ID NO: 18, or a variant thereof. In embodiments, the MDA-7/IL-24 protein includes an amino acid sequence that is about or at least about 80%, 85%, 90%, 95%, 96%, 97%, 98%, 99%, or 100% identical to SEQ ID NO: 18. In embodiments, the MDA-7/IL-24 protein includes an amino acid sequence that is about or at least about 90% identical to SEQ ID NO: 18. In embodiments, the MDA-7/IL-24 protein includes an amino acid sequence that is about or at least about 95% identical to SEQ ID NO: 18.

In embodiments, the MDA-7/IL-24 protein includes an amino acid sequence that is about or at least about 80% identical to SEQ ID NO: 18. In embodiments, the MDA-7/IL-24 protein includes an amino acid sequence that is about or at least about 85% identical to SEQ ID NO: 18. In embodiments, the MDA-7/IL-24 protein includes an amino acid sequence that is about or at least about 90% identical to SEQ ID NO: 18. In embodiments, the MDA-7/IL-24 protein includes an amino acid sequence that is about or at least about 95% identical to SEQ ID NO: 18. In embodiments, the MDA-7/IL-24 protein includes an amino acid sequence that is about or at least about 96% identical to SEQ ID NO: 18. In embodiments, the MDA-7/IL-24 protein includes an amino acid sequence that is about or at least about 97% identical to SEQ ID NO: 18. In embodiments, the MDA-7/IL-24 protein includes an amino acid sequence that is about or at least about 98% identical to SEQ ID NO: 18. In embodiments, the MDA-7/IL-24 protein includes an amino acid sequence that is about or at least about 99% identical to SEQ ID NO: 18. In embodiments, the MDA-7/IL-24 protein includes an amino acid sequence that is about 100% identical to SEQ ID NO: 18. In embodiments, the MDA-7/IL-24 protein includes an amino acid sequence that is about or at least about 80%, 85%, 90%, 95%, 96%, 97%, 98%, 99%, or 100% identical across 25, 50, 75, or 100 continuous amino acids of SEQ ID NO: 18.

In embodiments, the MDA-7/IL-24 protein includes an amino acid sequence of SEQ ID NO: 3, or a variant thereof. In embodiments, the MDA-7/IL-24 protein includes an amino acid sequence that is about or at least about 80%, 85%, 90%, 95%, 96%, 97%, 98%, 99%, or 100% identical to SEQ ID NO: 3. In embodiments, the MDA-7/IL-24 protein includes an amino acid sequence that is about or at least about 90% identical to SEQ ID NO: 3. In embodiments, the MDA-7/IL-24 protein includes an amino acid sequence that is about or at least about 95% identical to SEQ ID NO: 3.

In embodiments, the MDA-7/IL-24 protein includes an amino acid sequence that is about or at least about 80% identical to SEQ ID NO: 3. In embodiments, the MDA-7/IL-24 protein includes an amino acid sequence that is about or at least about 85% identical to SEQ ID NO: 3. In embodiments, the MDA-7/IL-24 protein includes an amino acid sequence that is about or at least about 90% identical to SEQ ID NO: 3. In embodiments, the MDA-7/IL-24 protein includes an amino acid sequence that is about or at least about 95% identical to SEQ ID NO: 3. In embodiments, the MDA-7/IL-24 protein includes an amino acid sequence that is about or at least about 96% identical to SEQ ID NO: 3. In embodiments, the MDA-7/IL-24 protein includes an amino acid sequence that is about or at least about 97% identical to SEQ ID NO: 3. In embodiments, the MDA-7/IL-24 protein includes an amino acid sequence that is about or at least about 98% identical to SEQ ID NO: 3. In embodiments, the MDA-7/IL-24 protein includes an amino acid sequence that is about or at least about 99% identical to SEQ ID NO: 3. In embodiments, the MDA-7/IL-24 protein includes an amino acid sequence that is about 100% identical to SEQ ID NO: 3. In embodiments, the MDA-7/IL-24 protein includes an amino acid sequence that is about or at least about 80%, 85%, 90%, 95%, 96%, 97%, 98%, 99%, or 100% identical across 50, 75, 100, or 150 continuous amino acids of SEQ ID NO: 3.

In embodiments, the MDA-7/IL-24 protein includes a lysine to arginine mutation corresponding to a change of K122R relative to SEQ ID NO: 2, a change of K73R relative to SEQ ID NO: 3, or a change of K19R relative to SEQ ID NO: 4. SEQ ID NO: 18 is an example of an amino acid sequence having a mutation of K122R relative to SEQ ID NO: 2. However, because SEQ ID NO: 18 represents a shorter sequence than SEQ ID NO: 2, the position of the mutation with respect to SEQ ID NO: 18 is amino acid 19. Nonetheless, optimal alignment between the two sequences shows that SEQ ID NO: 18 aligns to a portion within SEQ ID NO: 2 that is 100% identical except at position 19 of SEQ ID NO: 18, corresponding to position 122 of SEQ ID NO: 2. In addition, SEQ ID NO: 18 represents the result of a K19R mutation to SEQ ID NO: 4, as the two sequences are completely identical except at the mutant position.

In embodiments, the insulin signal peptide is a human insulin signal peptide. In embodiments, the insulin signal peptide includes an amino acid sequence of SEQ ID NO: 5, or a variant thereof. In embodiments, the insulin signal peptide includes an amino acid sequence that is at least about 80%, 85%, 90%, 95%, 96%, 97%, 98%, 99%, or 100% identical to SEQ ID NO: 5. In embodiments, the insulin signal peptide includes an amino acid sequence that is about or at least about 90% identical to SEQ ID NO: 5. In embodiments, the insulin signal peptide includes an amino acid sequence that is about or at least about 95% identical to SEQ ID NO: 5. In embodiments, the insulin signal peptide has 1, 2, 3, 4, or 5 amino acid substitutions with respect to SEQ ID NO: 5. In embodiments, the insulin signal peptide is joined to the MDA-7/IL-24 protein by a linker of about or more than about 1, 2, 3, 4, 5, 6, 7, 8, 9, 10, or more amino acids. In embodiments, the linker is about 1-10, 2-8, 3-7, or 4-6 amino acids in length. In embodiments, the insulin signal peptide is at the N-terminus of the fusion protein. In embodiments, the insulin signal peptide is within about 1, 2, 3, 4, 5, or more amino acids of the N-terminus of the fusion protein.

In embodiments, the insulin signal peptide includes an amino acid sequence that is about or at least about 80% identical to SEQ ID NO: 5. In embodiments, the insulin signal peptide includes an amino acid sequence that is about or at least about 85% identical to SEQ ID NO: 5. In embodiments, the insulin signal peptide includes an amino acid sequence that is about or at least about 90% identical to SEQ ID NO: 5. In embodiments, the insulin signal peptide includes an amino acid sequence that is about or at least about 95% identical to SEQ ID NO: 5. In embodiments, the insulin signal peptide includes an amino acid sequence that is about or at least about 96% identical to SEQ ID NO: 5. In embodiments, the insulin signal peptide includes an amino acid sequence that is about or at least about 97% identical to SEQ ID NO: 5. In embodiments, the insulin signal peptide includes an amino acid sequence that is about or at least about 98% identical to SEQ ID NO: 5. In embodiments, the insulin signal peptide includes an amino acid sequence that is about or at least about 99% identical to SEQ ID NO: 5. In embodiments, the insulin signal peptide includes an amino acid sequence that is about 100% identical to SEQ ID NO: 5. In embodiments, the insulin signal peptide includes an amino acid sequence that is about or at least about 80%, 85%, 90%, 95%, 96%, 97%, 98%, 99%, or 100% identical across 5, 10, 15, 20, or 24 continuous amino acids of SEQ ID NO: 5.

In embodiments, the inclusion of the insulin signal peptide in the fusion protein functions to increase the mRNA transcript level, protein level, mature protein level, mature protein fraction, secretion, and/or anti-cancer activity of the MDA-7/IL-24 protein. In embodiments, functions of the signal peptide are measured relative to a protein consisting of the amino acid sequence of SEQ ID NO: 2 (or a polynucleotide or vector encoding the same). In embodiments, functions of the signal peptide are measured relative to the corresponding MDA-7/IL-24 protein lacking the insulin signal peptide (or a polynucleotide or vector encoding the same). In embodiments, the mRNA transcript level, protein level, mature protein level, mature protein fraction, secretion, and/or anti-cancer activity of the MDA-7/IL-24 protein is increased by about or at least about 5%, 10%, 15%, 20%, 30%, 40%, 50%, 75%, 100%, 150%, 200% or more. In embodiments, the increase is about 5-200%, 10-150%, 20-100%, or 40-75%. In embodiments, the increase is at least about 5%. Relative changes effected by the insulin signal peptide can be measured in any suitable system, such as in cultured cells described herein.

In embodiments, the polynucleotide encoding the fusion protein includes a sequence described herein. In embodiments, the polynucleotide includes a nucleotide sequence of any one of SEQ ID NOs: 6, 10-12, or 17, or a variant thereof. In embodiments, the polynucleotide includes a nucleotide sequence that is at least about 80%, 85%, 90%, 95%, 96%, 97%, 98%, 99%, or 100% identical to any one of SEQ ID NOs: 6, 10-12, or 17. In embodiments, the polynucleotide includes a nucleotide sequence that is about or at least about 90% identical to any one of SEQ ID NOs: 6, 10-12, or 17. In embodiments, the polynucleotide includes a nucleotide sequence that is about or at least about 95% identical to any one of SEQ ID NOs: 6, 10-12, or 17. In embodiments, the polynucleotide includes a nucleotide sequence that is about or at least about 80% identical (e.g. 90%, 95%, or 100% identical) to SEQ ID NO: 17.

In embodiments, the polynucleotide encoding the fusion protein includes a nucleotide sequence that is about or at least about 80% identical to SEQ ID NO: 6. In embodiments, the polynucleotide includes a nucleotide sequence that is about or at least about 85% identical to SEQ ID NO: 6. In embodiments, the polynucleotide includes a nucleotide sequence that is about or at least about 90% identical to SEQ ID NO: 6. In embodiments, the polynucleotide includes a nucleotide sequence that is about or at least about 95% identical to SEQ ID NO: 6. In embodiments, the polynucleotide includes a nucleotide sequence that is about or at least about 96% identical to SEQ ID NO: 6. In embodiments, the polynucleotide includes a nucleotide sequence that is about or at least about 97% identical to SEQ ID NO: 6. In embodiments, the polynucleotide includes a nucleotide sequence that is about or at least about 98% identical to SEQ ID NO: 6. In embodiments, the polynucleotide includes a nucleotide sequence that is about or at least about 99% identical to SEQ ID NO: 6. In embodiments, the polynucleotide includes a nucleotide sequence that is about 100% identical to SEQ ID NO: 6. In embodiments, the polynucleotide includes a nucleotide sequence that is about or at least about 80%, 85%, 90%, 95%, 96%, 97%, 98%, 99%, or 100% identical across 50, 100, 150, 200, 250, 300, or more continuous nucleotides of SEQ ID NO: 6.

In embodiments, the polynucleotide encoding the fusion protein includes a nucleotide sequence that is about or at least about 80% identical to SEQ ID NO: 10. In embodiments, the polynucleotide includes a nucleotide sequence that is about or at least about 85% identical to SEQ ID NO: 10. In embodiments, the polynucleotide includes a nucleotide sequence that is about or at least about 90% identical to SEQ ID NO: 10. In embodiments, the polynucleotide includes a nucleotide sequence that is about or at least about 95% identical to SEQ ID NO: 10. In embodiments, the polynucleotide includes a nucleotide sequence that is about or at least about 96% identical to SEQ ID NO: 10. In embodiments, the polynucleotide includes a nucleotide sequence that is about or at least about 97% identical to SEQ ID NO: 10. In embodiments, the polynucleotide includes a nucleotide sequence that is about or at least about 98% identical to SEQ ID NO: 10. In embodiments, the polynucleotide includes a nucleotide sequence that is about or at least about 99% identical to SEQ ID NO: 10. In embodiments, the polynucleotide includes a nucleotide sequence that is about 100% identical to SEQ ID NO: 10. In embodiments, the polynucleotide includes a nucleotide sequence that is about or at least about 80%, 85%, 90%, 95%, 96%, 97%, 98%, 99%, or 100% identical across 50, 100, 150, 200, 250, 300, or more continuous nucleotides of SEQ ID NO: 10.

In embodiments, the polynucleotide encoding the fusion protein includes a nucleotide sequence that is about or at least about 80% identical to SEQ ID NO: 11. In embodiments, the polynucleotide includes a nucleotide sequence that is about or at least about 85% identical to SEQ ID NO: 11. In embodiments, the polynucleotide includes a nucleotide sequence that is about or at least about 90% identical to SEQ ID NO: 11. In embodiments, the polynucleotide includes a nucleotide sequence that is about or at least about 95% identical to SEQ ID NO: 11. In embodiments, the polynucleotide includes a nucleotide sequence that is about or at least about 96% identical to SEQ ID NO: 11. In embodiments, the polynucleotide includes a nucleotide sequence that is about or at least about 97% identical to SEQ ID NO: 11. In embodiments, the polynucleotide includes a nucleotide sequence that is about or at least about 98% identical to SEQ ID NO: 11. In embodiments, the polynucleotide includes a nucleotide sequence that is about or at least about 99% identical to SEQ ID NO: 11. In embodiments, the polynucleotide includes a nucleotide sequence that is about 100% identical to SEQ ID NO: 11. In embodiments, the polynucleotide includes a nucleotide sequence that is about or at least about 80%, 85%, 90%, 95%, 96%, 97%, 98%, 99%, or 100% identical across 50, 100, 150, 200, 250, 300, or more continuous nucleotides of SEQ ID NO: 11.

In embodiments, the polynucleotide encoding the fusion protein includes a nucleotide sequence that is about or at least about 80% identical to SEQ ID NO: 12. In embodiments, the polynucleotide includes a nucleotide sequence that is about or at least about 85% identical to SEQ ID NO: 12. In embodiments, the polynucleotide includes a nucleotide sequence that is about or at least about 90% identical to SEQ ID NO: 12. In embodiments, the polynucleotide includes a nucleotide sequence that is about or at least about 95% identical to SEQ ID NO: 12. In embodiments, the polynucleotide includes a nucleotide sequence that is about or at least about 96% identical to SEQ ID NO: 12. In embodiments, the polynucleotide includes a nucleotide sequence that is about or at least about 97% identical to SEQ ID NO: 12. In embodiments, the polynucleotide includes a nucleotide sequence that is about or at least about 98% identical to SEQ ID NO: 12. In embodiments, the polynucleotide includes a nucleotide sequence that is about or at least about 99% identical to SEQ ID NO: 12. In embodiments, the polynucleotide includes a nucleotide sequence that is about 100% identical to SEQ ID NO: 12. In embodiments, the polynucleotide includes a nucleotide sequence that is about or at least about 80%, 85%, 90%, 95%, 96%, 97%, 98%, 99%, or 100% identical across 50, 100, 150, 200, 250, 300, or more continuous nucleotides of SEQ ID NO: 12.

In embodiments, the polynucleotide encoding the fusion protein includes a nucleotide sequence that is about or at least about 80% identical to SEQ ID NO: 17. In embodiments, the polynucleotide includes a nucleotide sequence that is about or at least about 85% identical to SEQ ID NO: 17. In embodiments, the polynucleotide includes a nucleotide sequence that is about or at least about 90% identical to SEQ ID NO: 17. In embodiments, the polynucleotide includes a nucleotide sequence that is about or at least about 95% identical to SEQ ID NO: 17. In embodiments, the polynucleotide includes a nucleotide sequence that is about or at least about 96% identical to SEQ ID NO: 17. In embodiments, the polynucleotide includes a nucleotide sequence that is about or at least about 97% identical to SEQ ID NO: 17. In embodiments, the polynucleotide includes a nucleotide sequence that is about or at least about 98% identical to SEQ ID NO: 17. In embodiments, the polynucleotide includes a nucleotide sequence that is about or at least about 99% identical to SEQ ID NO: 17. In embodiments, the polynucleotide includes a nucleotide sequence that is about 100% identical to SEQ ID NO: 17. In embodiments, the polynucleotide includes a nucleotide sequence that is about or at least about 80%, 85%, 90%, 95%, 96%, 97%, 98%, 99%, or 100% identical across 50, 100, 150, 200, 250, 300, or more continuous nucleotides of SEQ ID NO: 17.

In embodiments, the MDA-7/IL-24 protein retains a biological activity. As a cytokine and a member of the IL-10 cytokine gene family, MDA-7/IL-24 natively signals through receptor dimers consisting of an R1 type receptor and an R2 type receptor (IL-20R1 and IL-20R2; IL-22R1 and IL-20R2; or a unique receptor pair IL-20R1 and IL-22R1) in order to activate downstream signaling events. Assays for measuring such activities are available (see, e.g., WO2018089995A1). In embodiments, an MDA-7/IL-24 protein is a variant, homolog, or isoform that retains at least 50%, 60%, 70%, 80%, 85%, 90%, 95%, 100%, or more of the biological activity of an MDA-7/IL-24 protein of SEQ ID NO: 2 or SEQ ID NO: 3. In embodiments, the MDA-7/IL-24 protein retains at least 80% of the biological activity of an MDA-7/IL-24 protein of SEQ ID NO: 3. In embodiments, the MDA-7/IL-24 protein retains at least 90% of the biological activity of an MDA-7/IL-24 protein of SEQ ID NO: 3. In embodiments, the MDA-7/IL-24 protein is capable of activating an IL-20/IL-22 receptor complex of a cancer cell of the subject, or of a reference cell line (e.g. DU-145 cells).

In embodiments, the native signal peptide of the MDA-7/IL-24 protein is recombinantly replaced with an insulin signal peptide. In such cases, the polynucleotide does not encode the native signal peptide of MDA-7/IL-24 protein. In embodiments, the polynucleotide does not encode amino acids 1-49 of SEQ ID NO: 2. In embodiments, the MDA-7/IL-24 protein expressed from the polynucleotide, after intracellular processing for secretion, is a mature MDA-7/IL-24 protein lacking the insulin signal peptide initially translated with the MDA-7/IL-24 protein. In embodiments, the MDA-7/IL-24 protein is a truncated form of MDA-7/IL-24 protein that retains biological activity. For example, the MDA-7/IL-24 protein may lack the first 54 amino acids of SEQ ID NO: 3.

In some aspects, the present disclosure provides vectors comprising any of the polynucleotides described herein. In embodiments, the vectors are expression vectors, such that the inserted polynucleotides are operatively linked to regulatory (e.g., transcriptional and/or translational control) sequences. In this context, the term “operatively linked” means that the polynucleotide of interest is inserted into the vector such that regulatory sequences within the vector serve their intended function of regulating the transcription and/or translation of the polynucleotide, such as when expressed in a cell. The vector and expression control sequences are chosen to be compatible with an intended host or target cell. Examples of regulatory sequences include, but are not limited to, promoters, enhancers and other expression control elements (e.g., polyadenylation signals). Non-limiting examples of regulatory sequences for use in expression a protein in a mammalian cell include: promoters and/or enhancers derived from cytomegalovirus (CMV), Simian Virus 40 (SV40), adenovirus, (e.g., the adenovirus major late promoter (AdMLP) and polyoma; nonviral regulatory sequences, such as the ubiquitin promoter or β-globin promoter; and sequences from different sources, such as the SRα promoter system, which contains sequences from the SV40 early promoter and the long terminal repeat of human T cell leukemia virus type 1.

In embodiments, the vector is a plasmid vector. In embodiments, the vector is a viral vector, such as an adenoviral vector (Ad), an associated-adenoviral vector (AAV), a lentiviral vector, a retroviral vector, a herpesvirus, a vaccinia virus, a genetically modified HIV, vesicular stomatitis virus, or other suitable viral vector. In embodiments, the virus is an adenovirus. A variety of suitable adenoviruses are available. Non-limiting examples of adenoviruses that may be used in the expression of an MDA-7/IL-24 protein include those described in WO2018089995A1, WO2017062708A1, US20180243382A1, US20160008413A1, and Dash et al., Cancer Res 2014;74:563-74. In embodiments, the virus (e.g., an adenovirus) is a replication incompetent adenovirus, such that viral replication in a target cell is diminished or eliminated relative to a corresponding wild-type virus. In embodiments, viral replication is under control of a cancer-specific promoter, such that viral replication is higher in cancer cells than in non-cancer cells. A non-limiting example of a cancer-specific promoter is the cancer-selective Progression Elevated Gene-3 (PEG-3) promoter. Adenoviral replication can be made cancer-specific by, for example, placing E1A and E1B genes under control of the PEG-3 promoter.

In some aspects, the present disclosure provides kits for use in methods disclosed herein. In embodiments, the use of a kit includes administration of the first microbubble composition, a first ultrasound administration directed to the target tissue that disrupts the first microbubbles, administration of the second microbubble composition after the first ultrasound administration, and a second ultrasound administration directed to the target tissue that disrupts the second microbubbles. In some embodiments, the use of a kit includes administration of the first microbubble composition and a first ultrasound administration directed to the target tissue that disrupts the first microbubbles. In some embodiments, the use of a kit includes administration of the second microbubble composition and a second ultrasound administration directed to the target tissue that disrupts the second microbubbles.

In embodiments, the administration of the second microbubble composition is within about 60 minutes, about 30 minutes, about 10 minutes, or about 5 minutes of administration of the first microbubble composition. In some embodiments, the administration is within about 60 minutes. In some embodiments, the administration is within about 30 minutes. In some embodiments, the administration is within about 10 minutes. In some embodiments, the administration is within about 5 minutes.

In embodiments, the target tissue comprises a tumor. In some embodiments, the tumor is a metastatic tumor. In embodiments, the tumor is located in the brain, a breast, a lung, the skin, the gastrointestinal system, a bone, a peritoneal cavity, pancreas, head, neck, oral cavity, spinal cord, or intestine of a subject. In some embodiments, the tumor is located in the brain. In some embodiments, the tumor is located in a breast. In some embodiments, the tumor is located in a lung. In some embodiments, the tumor is located in the gastrointestinal system. In some embodiments, the tumor is located in a bone. In some embodiments, the tumor is located in a peritoneal cavity. In some embodiments, the tumor is located in the pancreas. In some embodiments, the tumor is located in the intestine. In some embodiments, the tumor is located in the oral cavity. In some embodiments, the tumor is located in the spinal cord. In some embodiments, the tumor is located in the head. In some embodiments, the tumor is located in the neck. In some embodiments, the tumor is located in or on the skin.

In embodiments, the tumor comprises glioblastoma, melanoma, breast cancer, bone cancer, pancreatic cancer, liver cancer, colon cancer, oral cancer, head and neck cancer, spinal cord cancer, neuroblastoma, kidney cancer, or lung cancer. In some embodiments, the tumor is glioblastoma. In some embodiments, the tumor is melanoma. In some embodiments, the tumor is breast cancer. In some embodiments, the tumor is bone cancer. In some embodiments, the tumor is pancreatic cancer. In some embodiments, the tumor is liver cancer. In some embodiments, the tumor is colon cancer. In some embodiments, the tumor is oral cancer. In some embodiments, the tumor is head and neck cancer. In some embodiments, the tumor is spinal cord cancer. In some embodiments, the tumor is neuroblastoma. In some embodiments, the tumor is kidney cancer. In some embodiments, the tumor is lung cancer.

In embodiments, the target tissue that is located within the brain, pancreas, stomach, intestines, bones, skin, oral cavity, head, neck, spinal cord, lungs, kidney, or liver of the subject. In some embodiments, the target tissue is located within the brain. In some embodiments, the target tissue is located within the pancreas. In some embodiments, the target tissue is located within the stomach. In some embodiments, the target tissue is located within the intestines. In some embodiments, the target tissue is located within the bones. In some embodiments, the target tissue is located within the skin. In some embodiments, the target tissue is located within the oral cavity. In some embodiments, the target tissue is located within the head. In some embodiments, the target tissue is located within the neck. In some embodiments, the target tissue is located within the spinal cord. In some embodiments, the target tissue is located within the lungs. In some embodiments, the target tissue is located within the kidney. In some embodiments, the target tissue is located within the liver.

In embodiments, the administration of the first microbubble composition is in an amount effective to increase delivery of the active agent to the target tissue.

In embodiments, the administration of the first microbubble composition is in an amount effective to increase delivery of the active agent across the blood-brain barrier.

### Methods

In some aspects, the present disclosure provides methods of administering an active agent to a target tissue. In embodiments, the method comprises administering one or more compositions described herein, such as one or more compositions of a kit described herein. In embodiments, the active agent is a therapeutic agent or an imaging agent. In embodiments, the method includes: administering to a subject a first microbubble composition, the first microbubble composition containing first microbubbles and not containing the active agent; a first ultrasound administration directed to the target tissue that disrupts the first microbubbles; administering to the subject a second microbubble composition after the first ultrasound administration, the second microbubble composition containing second microbubbles complexed with the active agent; and a second ultrasound administration directed to the target tissue that disrupts the second microbubbles and releases the active agent to the target tissue.

In embodiments, the method includes administering the second microbubble composition within about 60 minutes, about 30 minutes, about 10 minutes, and about 5 minutes of administering the first microbubble composition. In some embodiments, the second microbubble composition is administered within about 60 minutes of administering the first microbubble composition. In some embodiments, the second microbubble composition is administered within about 30 minutes of administering the first microbubble composition. In some embodiments, the second microbubble composition is administered within about 10 minutes of administering the first microbubble composition. In some embodiments, the second microbubble composition is administered within about 5 minutes of administering the first microbubble composition.

In embodiments, the method includes administering the first microbubble composition, the second microbubble composition, or both by intravenous administration. In some embodiments, the first microbubble composition administration is by intravenous administration. In some embodiments, the second microbubble composition administration is by intravenous administration. In some embodiments, both the first microbubble and second microbubble compositions administrations are by intravenous administration.

In embodiments, the first and/or second microbubbles are microbubbles as described herein, such as with respect to the first and/or second microbubbles of a kit described herein. In embodiments, the first and/or second microbubbles have a mean or median diameter of about 1 micron to about 50 microns, about 1 micron to about 25 microns, about 1 micron to about 10 microns, or about 1 micron to about 5 microns. In embodiments, the first and/or second microbubbles have a mean or median diameter of about 1 micron to about 5 microns. In embodiments, the first and/or second microbubbles have a mean or medium diameter of about 2.5 microns to about 4 microns.

In embodiments, the first and/or second microbubbles comprise a targeting moiety. In some embodiments, the first microbubbles contain a targeting moiety. In some embodiments, the second microbubbles contain a targeting moiety. In some embodiments, the first and second microbubbles both contain a targeting moiety, which may be the same or different. In embodiments, the first and second microbubbles include a targeting moiety that binds the same target. In embodiments, the targeting moiety specifically binds a particular protein, a particular cell, or a particular tissue.

In embodiments, the targeting moiety comprises a VEGF polypeptide or single-chain variant thereof, which binds a VEGF receptor. In some embodiments, the targeting moiety is a VEGF polypeptide. In some embodiments, the targeting moiety is a single-chain variant of VEGF. Non-limiting examples of VEGF polypeptides and single-chain variants thereof useful as targeting moieties are provided in US20080312410A1, which is incorporated herein by reference.

In embodiments, the targeting moiety comprises a VCAM1 antibody or epitope-binding fragment thereof. In some embodiments, the targeting moiety is a VCAM1 antibody. In some embodiments, the targeting moiety is a VCAM1 epitope-binding fragment of an antibody.

In embodiments, the targeting moiety comprises a PSMA antibody or epitope-binding fragment thereof. In some embodiments, the targeting moiety is a PSMA antibody. In some embodiments, the targeting moiety is a PSMA epitope-binding fragment of an antibody.

In embodiments, the target tissue comprises a tumor. In some embodiments, the tumor is a metastatic tumor. In embodiments, the tumor is located in the brain, a breast, a lung, the skin, the gastrointestinal system, a bone, a peritoneal cavity, pancreas, head, neck, oral cavity, spinal cord, or intestine of a subject. In some embodiments, the tumor is located in the brain. In some embodiments, the tumor is located in a breast. In some embodiments, the tumor is located in a lung. In some embodiments, the tumor is located in the gastrointestinal system. In some embodiments, the tumor is located in a bone. In some embodiments, the tumor is located in a peritoneal cavity. In some embodiments, the tumor is located in the pancreas. In some embodiments, the tumor is located in the intestine. In some embodiments, the tumor is located in the oral cavity. In some embodiments, the tumor is located in the spinal cord. In some embodiments, the tumor is located in the head. In some embodiments, the tumor is located in the neck. In some embodiments, the tumor is located in or on the skin.

In embodiments, the tumor comprises glioblastoma, melanoma, breast cancer, bone cancer, pancreatic cancer, liver cancer, colon cancer, oral cancer, head and neck cancer, spinal cord cancer, neuroblastoma, kidney cancer, or lung cancer. In some embodiments, the tumor is glioblastoma. In some embodiments, the tumor is melanoma. In some embodiments, the tumor is breast cancer. In some embodiments, the tumor is bone cancer. In some embodiments, the tumor is pancreatic cancer. In some embodiments, the tumor is liver cancer. In some embodiments, the tumor is colon cancer. In some embodiments, the tumor is oral cancer. In some embodiments, the tumor is head and neck cancer. In some embodiments, the tumor is spinal cord cancer. In some embodiments, the tumor is neuroblastoma. In some embodiments, the tumor is kidney cancer. In some embodiments, the tumor is lung cancer.

In embodiments, the target tissue that is located within the brain, pancreas, stomach, intestines, bones, skin, oral cavity, head, neck, spinal cord, lungs, kidney, or liver of the subject. In some embodiments, the target tissue is located within the brain. In some embodiments, the target tissue is located within the pancreas. In some embodiments, the target tissue is located within the stomach. In some embodiments, the target tissue is located within the intestines. In some embodiments, the target tissue is located within the bones. In some embodiments, the target tissue is located within the skin. In some embodiments, the target tissue is located within the oral cavity. In some embodiments, the target tissue is located within the head. In some embodiments, the target tissue is located within the neck. In some embodiments, the target tissue is located within the spinal cord. In some embodiments, the target tissue is located within the lungs. In some embodiments, the target tissue is located within the kidney. In some embodiments, the target tissue is located within the liver.

In embodiments, the target tissue is in a subject. In embodiments, the subject is being treated for cancer. In some embodiments, the subject previously had cancer. In some embodiments, the subject previously went into remission from cancer.

In embodiments, the first microbubble composition is administered in an amount effective to increase delivery of the active agent to the target tissue, such as after a first administering the first microbubble composition and first ultrasound administration at the target tissue. In embodiments, the first microbubble composition is administered in an amount effective to increase delivery of the active agent across the blood-brain barrier, such as after a first administering the first microbubble composition and first ultrasound administration at the target tissue. In embodiments, the first microbubble composition is administered in an amount effective to increase delivery of the active agent to the pancreas, such as after a first administering the first microbubble composition and first ultrasound administration at the target tissue. In embodiments, the increase in delivery is with respect to administration of the second microbubble composition and second ultrasound administration in the absence of the first microbubble composition and first ultrasound administration. In embodiments, administration of the first microbubble composition and first ultrasound administration increases delivery of the active agent delivered by the second microbubble composition and second ultrasound administration by about or more than about 25%, 50%, 100%, 200%, 300%, or more. In embodiments, delivery is increased by about or more than about 50%. In embodiments, delivery is increased by about or more than about 100%. In embodiments, delivery is increased by about or more than about 200%.

In embodiments, the active agent is an active agent as described herein, such as with respect to a kit described herein. In embodiments, the active agent comprises a protein or nucleic acid. In some embodiments, the active agent is a protein. In some embodiments, the active agent is a nucleic acid. In some embodiments, the active agent is a nucleic acid. In some embodiments, the active agent is a short hairpin RNA (shRNA). In some embodiments, the active agent is a small interfering RNA (siRNA). In some embodiments, the active agent is an antisense RNA. In some embodiments, the active agent is DNA. In embodiments, the DNA encodes an shRNA or an antisense RNA. In embodiments, the active agent is an anti-cancer agent. In embodiments, the active agent inhibits the expression of MDA-9/Syntenin (an MDA-9/Syntenin inhibitor). In embodiments, the active agent is an MDA-7/IL-24 protein (e.g., a fusion protein) or variant thereof, a polynucleotide encoding the same, or a vector comprising such polynucleotide, non-limiting examples of which are described herein. In embodiments, the active agent comprises a virus, such as an adenovirus. In embodiments, replication of the virus is under control of a cancer-specific promoter.

In embodiments, administering a composition comprising the MDA-7/IL-24 protein comprises administering to a target tissue, such as to a tumor, a site from which a tumor has been surgically removed, and/or to a bone of a subject. In embodiments, administering to the target tissue comprises injection into or adjacent to the target tissue, or topical application to the target tissue. In embodiments, the composition is delivered distally to the target tissue, but is formulated to traffic the MDA-7/IL-24 protein (or polynucleotide or vector encoding the protein) to the target tissue. In embodiments, a moiety that traffics to a particular tissue, such as a cancer tissues and/or a bone tissue, is complexed with the MDA-7/IL-24 protein (or polynucleotide or vector encoding the protein). Complexing can be directly with the targeting moiety, such as a covalent or non-covalent interaction. Complexing can be indirect, such that the MDA-7/IL-24 protein (or polynucleotide or vector encoding the protein) and the targeting moiety are separated by one or more other molecules joining the two, via covalent or non-covalent interactions. In general, a targeting moiety is a moiety able to bind to or otherwise associate with a biological entity (e.g., a membrane component, a cell surface receptor, cell specific membrane antigen, or the like), with a higher affinity than one or more non-target biological entity (e.g., cell surface components of one or more different tissues). A targeting moiety typically allows a cargo (e.g., a polynucleotide, vector, or protein) to become localized at a particular targeting site to a higher degree than elsewhere in the body of the subject, or to a higher degree at the target site than would be accomplished in the absence of the targeting moiety. Non-limiting examples of targeting moieties include antibodies, antigen-binding antibody fragments, aptamers, peptides, hormones, growth factors, ligands (e.g., receptor ligands), small molecules, and the like. Illustrative examples of targeting moieties that traffic to bone are described in US20120028350A1, US20160052968A1, US20040038946A1, and US20180208650A1. In embodiments, the microbubbles are complexed with a targeting moiety that traffics the microbubbles to a particular tissue, such as a cancer tissue, cancer vasculature, or a bone tissue.

In embodiments, administering a composition comprising the MDA-9/Syntenin inhibitor comprises administering to a target tissue, such as to a tumor, a site from which a tumor has been surgically removed, and/or to a pancreas of a subject. In embodiments, administering to the target tissue comprises injection into or adjacent to the target tissue, or topical application to the target tissue. In embodiments, the composition is delivered distally to the target tissue, but is formulated to traffic the MDA-9/Syntenin inhibitor to the target tissue. In embodiments, a moiety that traffics to a particular tissue, such as a cancer tissues and/or pancreas tissue, is complexed with the MDA-9/Syntenin inhibitor. Complexing can be directly with the targeting moiety, such as a covalent or non-covalent interaction. Complexing can be indirect, such that the MDA-9/Syntenin inhibitor and the targeting moiety are separated by one or more other molecules joining the two, via covalent or non-covalent interactions. In general, a targeting moiety is a moiety able to bind to or otherwise associate with a biological entity (e.g., a membrane component, a cell surface receptor, cell specific membrane antigen, or the like), with a higher affinity than one or more non-target biological entity (e.g., cell surface components of one or more different tissues). A targeting moiety typically allows a cargo (e.g., a polynucleotide, vector, or protein) to become localized at a particular targeting site to a higher degree than elsewhere in the body of the subject, or to a higher degree at the target site than would be accomplished in the absence of the targeting moiety. Non-limiting examples of targeting moieties include antibodies, antigen-binding antibody fragments, aptamers, peptides, hormones, growth factors, ligands (e.g., receptor ligands), small molecules, and the like. In embodiments, the microbubbles are complexed with a targeting moiety that traffics the microbubbles to a particular tissue, such as a cancer tissue, cancer vasculature, or pancreatic tissue.

In embodiments, the method of administering an active or imaging agent to a target tissue comprises a third microbubbles composition. In embodiments, the third microbubbles composition is administered after the second ultrasound administration.

In embodiments, the method of administering an active or imaging agent to a target tissue comprises a fourth microbubbles composition. In embodiments, the fourth microbubbles composition is administered after the third ultrasound administration.

In embodiments, the third microbubbles composition comprises a second active agent. In embodiments, the fourth microbubbles composition comprises a third active agent. In some embodiments, the second active agent is the same as the first active agent. In some embodiments, the third active agent is the same as the first active agent. In some embodiments, the third active agent is the same as the second active agent.

In embodiments, the third microbubbles composition comprises a second imaging agent. In embodiments, the fourth microbubbles composition comprises a third imaging agent. In some embodiments, the second imaging agent is the same as the first imaging agent. In some embodiments, the third imaging agent is the same as the first imaging agent. In some embodiments, the third imaging agent is the same as the second imaging agent.

In embodiments, a microbubble composition is complexed with more than one active agent. In embodiments, a microbubble composition is complexed with more than one imaging agent. In embodiments, a microbubble composition is complexed with two active agents. In embodiments, a microbubble composition is complexed with two imaging agents. In embodiments, a microbubble composition is complexed with an active agent and an imaging agent.

In embodiments, is a method of treating a subject in need, wherein the method comprises administering an additional active or imaging agent that is not complexed with a microbubble.

In embodiments, is a method of treating a subject in need, wherein the method comprises administering an additional anti-cancer agent that is not complexed with a microbubble. In embodiments, the anti-cancer agent is selected from chemotherapy, hormonal therapy, radiotherapy, or immunotherapy. In some embodiments, the anti-cancer agent is chemotherapy. In some embodiments, the anti-cancer agent is hormonal therapy. In some embodiments, the anti-cancer agent is radiotherapy. In some embodiments, the anti-cancer agent is immunotherapy.

In embodiments, the anti-cancer agent is selected from, but not limited to, an alkylating agent, an antimetabolite, a natural product, a chemotherapeutic, a hormone, a polypeptide, or a small molecule having utility in methods of treating cancer. In some embodiments, the anti-cancer agent is an alkylating agent. In some embodiments, the anti-cancer agent is an antimetabolite. In some embodiments, the anti-cancer agent is a natural product. In some embodiments, the anti-cancer agent is a chemotherapeutic. In some embodiments, the anti-cancer agent is a hormone. In some embodiments, the anti-cancer agent is a polypeptide. In some embodiments, the anti-cancer agent is a small molecule having utility in methods of treating cancer.

In embodiments, the anti-cancer agent is gemcitabine. In embodiments, the anti-cancer agent is temozolomide.

In embodiments, the anti-cancer agent further comprises a pharmaceutically acceptable excipient.

### Experimental Protocols

### Microbubble and Active Agent/Imaging Agent Complex Formation:

Microbubbles (MBs) are reconstituted in buffer (e.g. PBS) containing an active agent (AA). The MBs and active agent are incubated for a period of time (e.g. 2 hours) at a certain temperature (e.g. 4° C.) After the incubation, unenclosed active agent is inactivated and/or removed. The MBs/AAs can be added to a suitable buffer (e.g. PBS) for administration to a subject.

### Generation of Focused Ultrasound (FUS) Waves and Treatment of Mice:

Focused ultrasound (FUS) utilizes the same concept of acoustic wave propagation as the more widely known diagnostic ultrasound applications. FUS can utilize concave transducers that have a single geometric focus or use phased arrays to electronically steer the ultrasound waves. The power of FUS is delivered during sonication, in order to induce mechanical effects, thermal effects, or both. The FUS transducer (e.g., 2.25 MHz, 0.50 in. Element Diameter, Standard Case Style, Straight UHF Connector, purchased from Olympus America Inc.) is used to perform sonication immediately following bubble administration (e.g., 15 seconds). The transducer is driven by a function generator (e.g., AGI-E4436B, Agilent Technologies, Palo Alto, Calif., USA) through a power amplifier (e.g., E&I 3100LA, ENI Inc., Rochester, N.Y., USA). A cone filled with degas sed and distilled water is attached to the transducer system. FUS is applied (e.g., 3.5 mV, 10 dB, 1 MHz) to a subject after microbubbles are adminstered.

### Intravenous FUS-DMB Subject Studies:

Diluted microbubbles (without an active agent or imaging agent) are injected into a subject (e.g. through the tail vein of a mouse) and allowed to circulate for a certain time (e.g. 15 sec). After circulating, the subject is sonicated (FUS) for a certain length of time (e.g., 1 minute) in a region of choice (e.g., the brain, the pancreas, the liver, the kidney). Optionally, the subject is injected with a second microbubble aliquot (with an active agent or imaging agent) and is sonicated for a certain length of time (e.g., 1 minute) after allowing the bubbles to circulate. Optionally, the subject is injected with a third microbubble aliquot (with an active agent or imaging agent) and is sonicated for a certain length of time (e.g., 1 minute) after allowing the bubbles to circulate. Optionally, the subject is injected with a fourth microbubble aliquot (with an active agent or imaging agent) and is sonicated for a certain length of time (e.g., 1 minute) after allowing the bubbles to circulate. Optionally, the subject can be injected with any number (>4) of microbubble aliquots (with an active agent or imaging agent) using the protocol described herein. The subject can be imaged using IVIS imager and followed for survival, toxicity, or effectiveness analysis.

### Intracranial FUS-DMB Subject Studies—Tumor Formation:

The subject is anesthetized and immobilized in a stereotactic frame. A needle attached to a syringe is inserted into the right basal ganglia with enough space for tumor cell accumulation. The entry point at the skull near the bregma. Intracerebral injection of cancer cells (e.g., 30,000 glioma cells) can be initiate formation of a tumor. The skull opening is enclosed with sterile bone wax, and the skin incision is closed using sterile surgical staples.

In some aspects, the present disclosure provides uses of a composition or kit described herein in the manufacture of a medicament for the treatment of cancer in a subject in need thereof. In embodiments, the composition includes a polynucleotide, vector, cell, or composition described herein.

SEQUENCES
(nucleotide sequence encoding an
MDA-7/IL-24 protein)
SEQ ID NO: 1
ATGAATTTTCAACAGAGGCTGCAAAGCCTGTGGAC
TTTAGCCAGACCCTTCTGCCCTCCTTTGCTGGCGA
CAGCCTCTCAAATGCAGATGGTTGTGCTCCCTTGC
CTGGGTTTTACCCTGCTTCTCTGGAGCCAGGTATC
AGGGGCCCAGGGCCAAGAATTCCACTTTGGGCCCT
GCCAAGTGAAGGGGGTTGTTCCCCAGAAACTGTGG
GAAGCCTTCTGGGCTGTGAAAGACACTATGCAAGC
TCAGGATAACATCACGAGTGCCCGGCTGCTGCAGC
AGGAGGTTCTGCAGAACGTCTCGGATGCTGAGAGC
TGTTACCTTGTCCACACCCTGCTGGAGTTCTACTT
GAAAACTGTTTTCAAAAACTACCACAATAGAACAG
TTGAAGTCAGGACTCTGAAGTCATTCTCTACTCTG
GCCAACAACTTTGTTCTCATCGTGTCACAACTGCA
ACCCAGTCAAGAAAATGAGATGTTTTCCATCAGAG
ACAGTGCACACAGGCGGTTCCTGCTATTCCGGAGA
GCATTTAAACAGTTGGACGTAGAAGCAGCTCTGAC
CAAAGCCCTTGGGGAAGTGGACATTCTTCTGACCT
GGATGCAGAAATTCTACAAGCTCTGA
(amino acid sequence of an
MDA-7/IL-24 protein)
SEQ ID NO: 2
MNFQQRLQSLWTLARPFCPPLLATASQMQMVVLPC
LGFTLLLWSQVSGAQGQEFHFGPCQVKGVVPQKLW
EAFWAVKDTMQAQDNITSARLLQQEVLQNVSDAES
CYLVHTLLEFYLKTVFKNYHNRTVEVRTLKSFSTL
ANNFVLIVSQLQPSQENEMFSIRDSAHRRFLLFRR
AFKQLDVEAALTKALGEVDILLTWMQKFYKL
(amino acid sequence of an
MDA-7/IL-24 protein)
SEQ ID NO: 3
QGQEFHFGPCQVKGVVPQKLWEAFWAVKDTMQAQD
NITSARLLQQEVLQNVSDAESCYLVHTLLEFYLKT
VFKNYHNRTVEVRTLKSFSTLANNFVLIVSQLQPS
QENEMFSIRDSAHRRFLLFRRAFKQLDVEAALTKA
LGEVDILLTWMQKFYKL
(amino acid sequence of an
MDA-7/IL-24 protein)
SEQ ID NO: 4
ESCYLVHTLLEFYLKTVFKNYHNRTVEVRTLKSFS
TLANNFVLIVSQLQPSQENEMFSIRDSAHRRFLLF
RRAFKQLDVEAALTKALGEVDILLTWMQKFYKL
(amino acid sequence of an
insulin signal peptide (sp))
SEQ ID NO: 5
MALWMRLLPLLALLALWGPDPAAA
(Encoding an insulin(sp)-MDA-7 sequence)
SEQ ID NO: 6
ATG GCG CTG TGG ATG CGC CTG CTG CCG
CTG CTG GCG CTG CTG GCG CTG TGG GGC
CCA GAT CCG GCG GCG GCG CAT CAC CAT
CAC CAT CAC GAG AAC CTG TAC TTC CAG
GGC ATG CAA GAA TTC CAC TTT GGG CCC
TGC CAA GTG AAG GGG GTT GTT CCC CAG
AAA CTG TGG GAA GCC TTC TGG GCT GTG
AAA GAC ACT ATG CAA GCT CAG GAT AAC
ATC ACG AGT GCC CGG CTG CTG CAG CAG
GAG GTT CTG CAG AAC GTC TCG GAT GCT
GAG AGC TGT TAC CTT GTC CAC ACC CTG
CTG GAG TTC TAC TTG AAA ACT GTT TTC
AAA AAC TAC CAC AAT AGA ACA GTT GAA
GTC AGG ACT CTG AAG TCA TTC TCT ACT
CTG GCC AAC AAC TTT GTT CTC ATC GTG
TCA CAA CTG CAA CCC AGT CAA GAA AAT
GAG ATG TTT TCC ATC AGA GAC AGT GCA
CAC AGG CGG TTC CTG CTA TTC CGG AGA
GCA TTC AAA CAG TTG GAC GTA GAA GCA
GCT CTG ACC AAA GCC CTT GGG GAA GTG
GAC ATT CTT CTG ACC TGG ATG CAG AAA
TTC TAC AAG CTC TAG
(Encoding a Flt3(sp)-MDA-7 sequence)
SEQ ID NO: 7
ATG ACA GTG CTG GCG CCA GCC TGG AGC
CCA ACA ACC TAT CTC CTC CTG CTG CTG
CTG CTG AGC GGA TCC ATG CAA GAA TTC
CAC TTT GGG CCC TGC CAA GTG AAG GGG
GTT GTT CCC CAG AAA CTG TGG GAA GCC
TTC TGG GCT GTG AAA GAC ACT ATG CAA
GCT CAG GAT AAC ATC ACG AGT GCC CGG
CTG CTG CAG CAG GAG GTT CTG CAG AAC
GTC TCG GAT GCT GAG AGC TGT TAC CTT
GTC CAC ACC CTG CTG GAG TTC TAC TTG
AAA ACT GTT TTC AAA AAC TAC CAC AAT
AGA ACA GTT GAA GTC AGG ACT CTG AAG
TCA TTC TCT ACT CTG GCC AAC AAC TTT
GTT CTC ATC GTG TCA CAA CTG CAA CCC
AGT CAA GAA AAT GAG ATG TTT TCC ATC
AGA GAC AGT GCA CAC AGG CGG TTT CTG
CTA TTC CGG AGA GCA TTC AAA CAG TTG
GAC GTA GAA GCA GCT CTG ACC AAA GCC
CTT GGG GAA GTG GAC ATT CTT CTG ACC
TGG ATG CAG AAA TTC TAC AAG CTC GGG
GGT TCT CAT CAT CAT CAT CAT CAT TGA
(Encoding a BM40(sp)-MDA-7 sequence)
SEQ ID NO: 8
ATG AGA GCC TGG ATC TTT TTT CTG CTC
TGC CTC GCT GGC AGA GCC CTG GCT CAT
CAC CAT CAC CAT CAC GAG AAC CTG TAC
TTC CAG GGC ATG CAA GAA TTC CAC TTT
GGG CCC TGC CAA GTG AAG GGG GTT GTT
CCC CAG AAA CTG TGG GAA GCC TTC TGG
GCT GTG AAA GAC ACT ATG CAA GCT CAG
GAT AAC ATC ACG AGT GCC CGG CTG CTG
CAG CAG GAG GTT CTG CAG AAC GTC TCG
GAT GCT GAG AGC TGT TAC CTT GTC CAC
ACC CTG CTG GAG TTC TAC TTG AAA ACT
GTT TTC AAA AAC TAC CAC AAT AGA ACA
GTT GAA GTC AGG ACT CTG AAG TCA TTC
TCT ACT CTG GCC AAC AAC TTT GTT CTC
ATC GTG TCA CAA CTG CAA CCC AGT CAA
GAA AAT GAG ATG TTT TCC ATC AGA GAC
AGT GCA CAC AGG CGG TTT CTG CTA TTC
CGG AGA GCA TTC AAA CAG TTG GAC GTA
GAA GCA GCT CTG ACC AAA GCC CTT GGG
GAA GTG GAC ATT CTT CTG ACC TGG ATG
CAG AAA TTC TAC AAG CTC TGA
(Encoding an IL-2(sp)-MDA-7 sequence)
SEQ ID NO: 9
ATG CAG CTG CTG TCA TGC ATC GCA TTG
ATC TTG GCG CTG GTG ATG CAA GAA TTC
CAC TTT GGG CCC TGC CAA GTG AAG GGG
GTT GTT CCC CAG AAA CTG TGG GAA GCC
TTC TGG GCT GTG AAA GAC ACT ATG CAA
GCT CAG GAT AAC ATC ACG AGT GCC CGG
CTG CTG CAG CAG GAG GTT CTG CAG AAC
GTC TCG GAT GCT GAG AGC TGT TAC CTT
GTC CAC ACC CTG CTG GAG TTC TAC TTG
AAA ACT GTT TTC AAA AAC TAC CAC AAT
AGA ACA GTT GAA GTC AGG ACT CTG AAG
TCA TTC TCT ACT CTG GCC AAC AAC TTT
GTT CTC ATC GTG TCA CAA CTG CAA CCC
AGT CAA GAA AAT GAG ATG TTT TCC ATC
AGA GAC AGT GCA CAC AGG CGG TTT CTG
CTA TTC CGG AGA GCA TTC AAA CAG TTG
GAC GTA GAA GCA GCT CTG ACC AAA GCC
CTT GGG GAA GTG GAC ATT CTT CTG ACC
TGG ATG CAG AAA TTC TAC AAG CTC TGA
(Encoding an MDA-7(K122R) sequence)
SEQ ID NO: 10
ATG AAT TTT CAA CAG AGG CTG CAA AGC
CTG TGG ACT TTA GCC AGA CCC TTC TGC
CCT CCT TTG CTG GCG ACA GCC TCT CAA
ATG CAG ATG GTT GTG CTC CCT TGC CTG
GGT TTT ACC CTG CTT CTC TGG AGC CAG
GTA TCA GGG GCC CAG GGC CAA GAA TTC
CAC TTT GGG CCC TGC CAA GTG AAG GGG
GTT GTT CCC CAG AAA CTG TGG GAA GCC
TTC TGG GCT GTG AAA GAC ACT ATG CAA
GCT CAG GAT AAC ATC ACG AGT GCC CGG
CTG CTG CAG CAG GAG GTT CTG CAG AAC
GTC TCG GAT GCT GAG AGC TGT TAC CTT
GTC CAC ACC CTG CTG GAG TTC TAC TTG
AAA ACT GTT TTC AGA AAC TAC CAC AAT
AGA ACA GTT GAA GTC AGG ACT CTG AAG
TCA TTC TCT ACT CTG GCC AAC AAC TTT
GTT CTC ATC GTG TCA CAA CTG CAA CCC
AGT CAA GAA AAT GAG ATG TTT TCC ATC
AGA GAC AGT GCA CAC AGG CGG TTT CTG
CTA TTC CGG AGA GCA TTC AAA CAG TTG
GAC GTA GAA GCA GCT CTG ACC AAA GCC
CTT GGG GAA GTG GAC ATT CTT CTG ACC
TGG ATG CAG AAA TTC TAC
(Encoding an insulin(sp)-MDA-7-K122R)
SEQ ID NO: 11
AAG CTC TGA
ATG GCG CTG TGG ATG CGC CTG CTG CCG
CTG CTG GCG CTG CTG GCG CTG TGG GGC
CCA GAT CCG GCG GCG GCG CAT CAC CAT
CAC CAT CAC GAG AAC CTG TAC TTC CAG
GGC ATG CAA GAA TTC CAC TTT GGG CCC
TGC CAA GTG AAG GGG GTT GTT CCC CAG
AAA CTG TGG GAA GCC TTC TGG GCT GTG
AAA GAC ACT ATG CAA GCT CAG GAT AAC
ATC ACG AGT GCC CGG CTG CTG CAG CAG
GAG GTT CTG CAG AAC GTC TCG GAT GCT
GAG AGC TGT TAC CTT GTC CAC ACC CTG
CTG GAG TTC TAC TTG AAA ACT GTT TTC
AGA AAC TAC CAC AAT AGA ACA GTT GAA
GTC AGG ACT CTG AAG TCA TTC TCT ACT
CTG GCC AAC AAC TTT GTT CTC ATC GTG
TCA CAA CTG CAA CCC AGT CAA GAA AAT
GAG ATG TTT TCC ATC AGA GAC AGT GCA
CAC AGG CGG TTC CTG CTA TTC CGG AGA
GCA TTC AAA CAG TTG GAC GTA GAA GCA
GCT CTG ACC AAA GCC CTT GGG GAA GTG
GAC ATT CTT CTG ACC TGG ATG CAG AAA
TTC TAC AAG CTC TAG
(Encoding an Insu1in(sp)-M4)
SEQ ID NO: 12
ATG GCG CTG TGG ATG CGC CTG CTG CCG
CTG CTG GCG CTG CTG GCG CTG TGG GGC
CCA GAT CCG GCG GCG GCG CAT CAC CAT
CAC CAT CAC GAG AAC CTG TAC TTC CAG
GGC ATG GAG AGC TGT TAC CTT GTC CAC
ACC CTG CTG GAG TTC TAC TTG AAA ACT
GTT TTC AAA AAC TAC CAC AAT AGA ACA
GTT GAA GTC AGG ACT CTG AAG TCA TTC
TCT ACT CTG GCC AAC AAC TTT GTT CTC
ATC GTG TCA CAA CTG CAA CCC AGT CAA
GAA AAT GAG ATG TTT TCC ATC AGA GAC
AGT GCA CAC AGG CGG TTC CTG CTA TTC
CGG AGA GCA TTC AAA CAG TTG GAC GTA
GAA GCA GCT CTG ACC AAA GCC CTT GGG
GAA GTG GAC ATT CTT CTG ACC TGG ATG
CAG AAA TTC TAC AAG CTC TAG
(Encoding an IL2(sp)-M4)
SEQ ID NO: 13
ATG CAG CTG CTG TCA TGC ATC GCA TTG
ATC TTG GCG CTG GTG ATG GAG AGC TGT
TAC CTT GTC CAC ACC CTG CTG GAG TTC
TAC TTG AAA ACT GTT TTC AAA AAC TAC
CAC AAT AGA ACA GTT GAA GTC AGG ACT
CTG AAG TCA TTC TCT ACT CTG GCC AAC
AAC TTT GTT CTC ATC GTG TCA CAA CTG
CAA CCC AGT CAA GAA AAT GAG ATG TTT
TCC ATC AGA GAC AGT GCA CAC AGG CGG
TTT CTG CTA TTC CGG AGA GCA TTC AAA
CAG TTG GAC GTA GAA GCA GCT CTG ACC
AAA GCC CTT GGG GAA GTG GAC ATT CTT
CTG ACC TGG ATG CAG AAA TTC TAC AAG
CTC TGA
(Encoding a Flt-3(sp)-M4)
SEQ ID NO: 14
ATG ACA GTG CTG GCG CCA GCC TGG AGC
CCA ACA ACC TAT CTC CTC CTG CTG CTG
CTG CTG AGC GGA TCC GAG AGC TGT TAC
CTT GTC CAC ACC CTG CTG GAG TTC TAC
TTG AAA ACT GTT TTC AAA AAC TAC CAC
AAT AGA ACA GTT GAA GTC AGG ACT CTG
AAG TCA TTC TCT ACT CTG GCC AAC AAC
TTT GTT CTC ATC GTG TCA CAA CTG CAA
CCC AGT CAA GAA AAT GAG ATG TTT TCC
ATC AGA GAC AGT GCA CAC AGG CGG TTT
CTG CTA TTC CGG AGA GCA TTC AAA CAG
TTG GAC GTA GAA GCA GCT CTG ACC AAA
GCC CTT GGG GAA GTG GAC ATT CTT CTG
ACC TGG ATG CAG AAA TTC TAC AAG CTC
GGG GGT TCT CAT CAT CAT CAT CAT CAT
TGA
(Encoding an MDA-7(sp)-M4)
SEQ ID NO: 15
ATG AAT TTT CAA CAG AGG CTG CAA AGC
CTG TGG ACT TTA GCC AGA CCC TTC TGC
CCT CCT TTG CTG GCG ACA GCC TCT CAA
ATG CAG ATG GTT GTG CTC CCT TGC CTG
GGT TTT ACC CTG CTT CTC TGG AGC CAG
GTA TCA GGG GCC CAG GGC GGA TCC GAG
AGC TGT TAC CTT GTC CAC ACC CTG CTG
GAG TTC TAC TTG AAA ACT GTT TTC AAA
AAC TAC CAC AAT AGA ACA GTT GAA GTC
AGG ACT CTG AAG TCA TTC TCT ACT CTG
GCC AAC AAC TTT GTT CTC ATC GTG TCA
CAA CTG CAA CCC AGT CAA GAA AAT GAG
ATG TTT TCC ATC AGA GAC AGT GCA CAC
AGG CGG TTT CTG CTA TTC CGG AGA GCA
TTC AAA CAG TTG GAC GTA GAA GCA GCT
CTG ACC AAA GCC CTT GGG GAA GTG GAC
ATT CTT CTG ACC TGG ATG CAG AAA TTC
TAC AAG CTC TGA
(Encoding a Flt-3(sp)-M4(K122R))
SEQ ID NO: 16
ATG ACA GTG CTG GCG CCA GCC TGG AGC
CCA ACA ACC TAT CTC CTC CTG CTG CTG
CTG CTG AGC GGA TCC GAG AGC TGT TAC
CTT GTC CAC ACC CTG CTG GAG TTC TAC
TTG AAA ACT GTT TTC AGA AAC TAC CAC
AAT AGA ACA GTT GAA GTC AGG ACT CTG
AAG TCA TTC TCT ACT CTG GCC AAC AAC
TTT GTT CTC ATC GTG TCA CAA CTG CAA
CCC AGT CAA GAA AAT GAG ATG TTT TCC
ATC AGA GAC AGT GCA CAC AGG CGG TTT
CTG CTA TTC CGG AGA GCA TTC AAA CAG
TTG GAC GTA GAA GCA GCT CTG ACC AAA
GCC CTT GGG GAA GTG GAC ATT CTT CTG
ACC TGG ATG CAG AAA TTC TAC AAG CTC
GGG GGT TCT CAT CAT CAT CAT CAT CAT
TGA
(Encoding an insulin(sp)-M4(K122R))
SEQ ID NO: 17
ATG GCG CTG TGG ATG CGC CTG CTG CCG
CTG CTG GCG CTG CTG GCG CTG TGG GGC
CCA GAT CCG GCG GCG GCG CAT CAC CAT
CAC CAT CAC GAG AAC CTG TAC TTC CAG
GGC ATG GAG AGC TGT TAC CTT GTC CAC
ACC CTG CTG GAG TTC TAC TTG AAA ACT
GTT TTC AGA AAC TAC CAC AAT AGA ACA
GTT GAA GTC AGG ACT CTG AAG TCA TTC
TCT ACT CTG GCC AAC AAC TTT GTT CTC
ATC GTG TCA CAA CTG CAA CCC AGT CAA
GAA AAT GAG ATG TTT TCC ATC AGA GAC
AGT GCA CAC AGG CGG TTC CTG CTA TTC
CGG AGA GCA TTC AAA CAG TTG GAC GTA
GAA GCA GCT CTG ACC AAA GCC CTT GGG
GAA GTG GAC ATT CTT CTG ACC TGG ATG
CAG AAA TTC TAC AAG CTC TAG
(amino acid sequence of an MDA-7/
IL-24 protein (K122R))
SEQ ID NO: 18
ESCYLVHTLLEFYLKTVFRNYHNRTVEVRTLKSFS
TLANNFVLIVSQLQPSQENEMFSIRDSAHRRFLLF
RRAFKQLDVEAALTKALGEVDILLTWMQKFYKL
(Specific hairpin small interfering
(siRNA) oligonucleotides used for
Ad.shMDA-9), sense strand
SEQ ID NO: 19
5′-GATCCGCGGATGGCACCAAGCATTTTCAAGAG
AAATGCTTGGTGCCATCCGCTTTTTTGGAAA-3′
(Specific hairpin small interfering
(siRNA) oligonucleotides used for
Ad.shMDA-9), antisense strand
SEQ ID NO: 20
5′-AGCTTTTCCAAAAAAGCGGATGGCACCAAGCA
TTTCTCTTGAAAATGCTTGGTGCCATCCGCG-3′

### P EMBODIMENTS

1. A method of administering an active agent to a target tissue, wherein the active agent is a therapeutic agent or an imaging agent, the method comprising:
(a) administering to a subject a first microbubble composition, the first microbubble composition comprising first microbubbles and not comprising the active agent;
(b) a first ultrasound administration directed to the target tissue that disrupts the first microbubbles;
(c) administering to the subject a second microbubble composition after the first ultrasound administration, the second microbubble composition comprising second microbubbles complexed with the active agent; and
(d) a second ultrasound administration directed to the target tissue that disrupts the second microbubbles and releases the active agent to the target tissue.

2. The method of embodiment P1, wherein the second microbubble composition is administered within about 60 minutes, 30 minutes, 10 minutes, or 5 minutes of administering the first microbubble composition.

3. The method of embodiment P1 or P2, wherein administering the first microbubble composition, the second microbubble composition, or both comprises intravenous administration.
4. The method of any one of embodiments P1-P3, wherein the first and/or second microbubbles have a mean or median diameter of about 1 micron to about 5 microns, or about 2.5 microns to about 4 microns.
5. The method of any one of embodiments P1-P4, wherein the first and/or second microbubbles comprise a targeting moiety.
6. The method of embodiment P5, wherein the targeting moiety comprises (a) a VEGF polypeptide or single-chain variant thereof, (b) a VCAM1 antibody or epitope-binding fragment thereof, or (c) a PSMA antibody or epitope-binding fragment thereof.
7. The method of any one of embodiments P1-P8, wherein the target tissue comprises a tumor.
8. The method of embodiment P9, wherein the tumor is a metastatic tumor.
9. The method of embodiment P9 or P10, wherein the tumor is located in a brain, a breast, a lung, a gastrointestinal system, a bone, a peritoneal cavity, pancreas, or intestine of the subject.
10. The method of any one of embodiments P9-P11, wherein the tumor comprises glioblastoma, melanoma, breast cancer, or lung cancer.
11. The method of any one of embodiments P1-P12, wherein the target tissue is located within the brain, pancreas, stomach, intestines, bones, or liver of the subject.
12. The method of any one of embodiments P1-P13, wherein the target tissue is in the brain of the subject.
13. The method of any one of embodiments P1-P14, wherein the first microbubble composition is administered in an amount effective to increase delivery of the active agent to the target tissue.
14. The method of embodiment P16, wherein the first microbubble composition is administered in an amount effective to increase delivery of the active agent across the blood-brain barrier.
15. The method of any one of embodiments P1-P17, wherein the active agent comprises a protein or a nucleic acid, optionally wherein the nucleic acid comprises an shRNA, an siRNA, RNA, or DNA.
16. The method of any one of embodiments P1-P17, wherein the active agent comprises an anti-cancer agent.
17. The method of any one of embodiments P1-P17, wherein the active agent comprises a virus.
18. The method of embodiment P21, wherein the virus is an adenovirus.
19. The method of embodiment P21 or P22, wherein replication of the virus is under control of a cancer-specific promoter.
20. The method of any one of embodiments P21-P23, wherein the virus comprises a polynucleotide encoding an shRNA, an siRNA, or an antisense RNA.
21. The method of any one of embodiments P21-P23, wherein the virus comprises a polynucleotide encoding an MDA-7/IL-24 protein.
22. The method of any one of embodiments P1-P17, wherein the active agent comprises an MDA-7/IL-24 protein or a polynucleotide encoding the MDA-7/IL-24 protein.
23. The method of embodiment P25 or P26, wherein the MDA-7/IL-24 protein is a fusion protein.
24. The method of any one embodiments P25-P27, wherein the MDA-7/IL-24 protein comprises an insulin signal peptide.
25. The method of any one of embodiments P25-P28, wherein the MDA-7/IL-24 protein comprises an amino acid sequence that is at least 90% identical to SEQ ID NO: 3 or 4.
26. The method of any one of embodiments P25-P29, wherein the MDA-7/IL-24 protein comprises a mutation corresponding to (a) a change of K73R relative to SEQ ID NO: 3, or (b) a change of K19R relative to SEQ ID NO: 4.
27. A kit for use in the treatment of a target tissue with an active agent, the kit comprising a first and second microbubble composition, wherein (i) the active agent is a therapeutic agent or an imaging agent, (ii) the first microbubble composition comprises first microbubbles and does not comprise the active agent, and (iii) the second microbubble composition comprises second microbubbles complexed with the active agent.
28. The kit of embodiment P58, wherein the first microbubble composition, the second microbubble composition, or both are formulated for intravenous administration.
29. The kit of embodiment P58 or P59, wherein the first and/or second microbubbles have a mean or median diameter of about 1 micron to about 5 microns, or about 2.5 microns to about 4 microns.
30. The kit of any one of embodiments P58-P60, wherein the first and/or second microbubbles comprise a targeting moiety.
31. The kit of embodiment P61, wherein the targeting moiety comprises (a) a VEGF polypeptide or single-chain variant thereof, (b) a VCAM1 antibody or epitope-binding fragment thereof, or (c) a PSMA antibody or epitope-binding fragment thereof.
32. The kit of any one of embodiments P58-P63, wherein the active agent comprises a protein or a nucleic acid, optionally wherein the nucleic acid comprises an shRNA, an siRNA, RNA, or DNA.
33. The kit of any one of embodiments P58-P63, wherein the active agent comprises an anti-cancer agent.
34. The kit of any one of embodiments P58-P63, wherein the active agent comprises a virus.
35. The kit of embodiment P66, wherein the virus is an adenovirus.
36. The kit of embodiment P66 or P67, wherein replication of the virus is under control of a cancer-specific promoter.
37. The kit of any one of embodiments P66-P68, wherein the virus comprises a polynucleotide encoding an shRNA, an siRNA, or an antisense RNA.
38. The kit of any one of embodiments P66-P68, wherein the virus comprises a polynucleotide encoding an MDA-7/IL-24 protein.
39. The kit of any one of embodiments P58-P63, wherein the active agent comprises an MDA-7/IL-24 protein or a polynucleotide encoding the MDA-7/IL-24 protein.
40. The kit of embodiment P70 or P71, wherein the MDA-7/IL-24 protein is a fusion protein.
41. The kit of any one of embodiments P70-P72, wherein the MDA-7/IL-24 protein comprises an insulin signal peptide.
42. The kit of any one of embodiments P70-P73, wherein the MDA-7/IL-24 protein comprises an amino acid sequence that is at least 90% identical to SEQ ID NO: 3 or 4.
43. The kit of any one of embodiments P70-P74, wherein the MDA-7/IL-24 protein comprises a mutation corresponding to (a) a change of K73R relative to SEQ ID NO: 3, or (b) a change of K19R relative to SEQ ID NO: 4.

### N EMBODIMENTS

1. A method of administering an active agent to a target tissue, wherein the active agent is a therapeutic agent or an imaging agent, the method comprising:
(a) administering to a subject a first microbubble composition, the first microbubble composition comprising first microbubbles and not comprising the active agent;
(b) a first ultrasound administration directed to the target tissue that disrupts the first microbubbles;
(c) administering to the subject a second microbubble composition after the first ultrasound administration, the second microbubble composition comprising second microbubbles complexed with the active agent; and
(d) a second ultrasound administration directed to the target tissue that disrupts the second microbubbles and releases the active agent to the target tissue.
   2. The method of embodiment N1, wherein the second microbubble composition is administered within about 60 minutes, 30 minutes, 10 minutes, or 5 minutes of administering the first microbubble composition.
   3. The method of embodiment N1 or N2, wherein administering the first microbubble composition, the second microbubble composition, or both comprises intravenous administration.
   4. The method of any one of embodiments N1-N3, wherein the first and/or second microbubbles have a mean or median diameter of about 1 micron to about 5 microns, or about 2.5 microns to about 4 microns.
   5. The method of any one of embodiment N1-N4, wherein the first and/or second microbubbles comprise a targeting moiety.
   6. The method of embodiment N5, wherein the targeting moiety is a molecule selected from an antibody, antibody fragment, a binding protein, a binding protein fragment, a receptor, a receptor fragment, a receptor ligand, a peptide, a polypeptide, a polynucleic acid, a polysaccharide, a lipid, a polymer, a tumor associated antigen, a tissue type-associated antigen, a vascular associated antigen or any combination of molecules thereof.
   7. The method of embodiment N6, wherein the targeting moiety binds a tissue specific antigen or a tumor associated antigen.
   8. The method of embodiment N6, wherein the targeting moiety comprises (a) a VEGF polypeptide or single-chain variant thereof, (b) a VCAM1 antibody or epitope-binding fragment thereof, or (c) a PSMA antibody or epitope-binding fragment thereof.
   9. The method of any one of embodiments N1-N8, wherein the target tissue comprises a tumor.
   10. The method of embodiment N9, wherein the tumor is a metastatic tumor.
   11. The method of embodiment N9 or N10, wherein the tumor is located in the brain, a breast, a lung, the gastrointestinal system, a bone, the peritoneal cavity, the oral cavity, pancreas, intestine, skin, head, neck, spinal cord, or liver of the subject.
   12. The method of any one of embodiments N9-N11, wherein the tumor comprises glioblastoma, melanoma, breast cancer, bone cancer, pancreatic cancer, liver cancer, colon cancer, oral cancer, head and neck cancer, spinal cord cancer, neuroblastoma, kidney cancer, or lung cancer.
   13. The method of any one of embodiments N1-N12, wherein the target tissue is located within the brain, pancreas, stomach, intestines, bones, skin, oral cavity, head, neck, spinal cord, lungs, kidney, or liver of the subject.
   14. The method of embodiment N13, wherein the target tissue is in the brain of the subject.
15. The method of embodiment N13, wherein the target tissue is in the pancreas of the subject.
16. The method of any one of embodiments N1-N15, wherein the first microbubble composition is administered in an amount effective to increase delivery of the active agent to the target tissue.
   17. The method of embodiment N16, wherein the first microbubble composition is administered in an amount effective to increase delivery of the active agent across the blood-brain barrier.
   18. The method of any one of embodiments N1-N17, wherein the active agent comprises a protein or a nucleic acid, optionally wherein the nucleic acid comprises an shRNA, an siRNA, an miRNA, an lncRNA, an mRNA, RNA, a vector, a plasmid, DNA, or any combination thereof.
   19. The method of any one of embodiments N1-N17, wherein the active agent comprises an anti-cancer agent.
   20. The method of embodiment N19, wherein the anti-cancer agent is selected from an alkylating agent, an antimetabolite, a natural product, chemotherapeutic, hormone, polypeptide, or a small molecule having utility in methods of treating cancer.
   21. The method of any one of embodiments N1-N17, wherein the active agent comprises a virus.
   22. The method of embodiment N21, wherein the virus is selected from an adenovirus, a tropism modified adenovirus, a cancer terminator virus (CTV), a lentivirus, a retrovirus, a herpesvirus, a vaccinia virus, a genetically modified HIV, a tripartite theranostic cancer terminator virus (TCTV), an avian associated virus (AAV), and/or a vesicular stomatitis virus.
   23. The method of embodiment N21 or N22, wherein replication of the virus is under control of a cancer-selective promoter.
   24. The method of any one of embodiments N21-N23, wherein the virus comprises a polynucleotide encoding an shRNA, an siRNA, an miRNA, a sense RNA, an antisense RNA or lncRNA.
   25. The method of any one of embodiments N21-N23, wherein the virus comprises a polynucleotide encoding an MDA-7/IL-24 protein.
   26. The method of any one of embodiments N1-N17, wherein the active agent comprises an MDA-7/IL-24 protein or a polynucleotide encoding the MDA-7/IL-24 protein.
   27. The method of embodiment N25 or N26, wherein the MDA-7/IL-24 protein is a fusion protein.
   28. The method of any one of embodiments N25-N27, wherein the MDA-7/IL-24 protein comprises an insulin signal peptide.
   29. The method of any one of embodiments N25-N27, wherein the MDA-7/IL-24 protein comprises an amino acid sequence that is at least 90% identical to SEQ ID NO: 3 or 4.
   30. The method of any one of embodiments N25-N28, wherein the MDA-7/IL-24 protein comprises a mutation corresponding to (a) a change of K122R relative to SEQ ID NO: 2 (b) a change of K73R relative to SEQ ID NO: 3, (c) a change of K19R relative to SEQ ID NO: 4, or (d) SEQ ID NO: 18.
   31. The method of any one of embodiments N1-N17, wherein the active agent comprises an MDA-9/Syntenin inhibitor.
   32. The method of any one of embodiments N21-N23, wherein the virus comprises a polynucleotide encoding an MDA-9/Syntenin inhibitor.
   33. The method of embodiment N32, wherein the polynucleotide sequence comprises a sequence that encodes an MDA-9/Syntenin siRNA, an shRNA, a miRNA, lncRNA, or antisense RNA sequence.
   34. The method of embodiment N33, wherein the sequence comprises SEQ ID NO: 20.
   35. The method of any one of embodiments N1-N34, further comprising:
(e) administering to the subject a third microbubble composition after the second ultrasound administration, the third microbubble composition comprising second microbubbles complexed with a second active agent or imaging agent; and
(f) a third ultrasound administration directed to the target tissue that disrupts the third microbubbles and releases the second active agent or imaging agent to the target tissue.
   36. The method of embodiment N35, wherein the second active agent or imaging agent is the same as the first active agent or imaging agent.
   37. The method of any one of embodiments N35-N36, further comprising:
(g) administering to the subject a fourth microbubble composition after the third ultrasound administration, the fourth microbubble composition comprising third microbubbles complexed with a third active agent or imaging agent; and
(h) a fourth ultrasound administration directed to the target tissue that disrupts the fourth microbubbles and releases the third active agent or imaging agent to the target tissue.
   38. The method of embodiment N37, wherein the third active agent or imaging agent is the same as the first active agent or imaging agent.
   39. The method of embodiment N37, wherein the third active agent or imaging agent is the same as the second active agent or imaging agent.
   40. The method of embodiment N37, wherein the third active agent or imaging agent is the same as the first and second active agent or imaging agent.
   41. The method of any one of embodiments N1-N40, wherein the microbubble composition comprises two or more active agents.
   42. The method of any of embodiments N1-N41, wherein the subject has cancer or is at risk of having cancer.
   43. The method of embodiment N42, wherein the cancer is a solid tumor cancer.
   44. The method of embodiment N43, wherein the cancer is brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's Disease, and Non-Hodgkin's lymphoma, thyroid cancer, endocrine system cancer, breast cancer, cervical cancer, colon cancer, head and neck cancer, liver cancer, kidney cancer, stomach cancer, uterine cancer, thyroid carcinoma, cholangiocarcinoma, pancreatic adenocarcinoma, pancreatic ductal adenocarcinoma (PDAC), skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, rhabdomyosarcoma, primary thrombocytosis, primary macroglobulinemia, primary brain tumors, malignant pancreatic insulinoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.
   45. The method of embodiment N42, wherein the cancer is brain cancer.
   46. The method of embodiment N42, wherein the cancer is pancreatic cancer.
   47. The method of embodiment N42, wherein the cancer is metastatic cancer.
   48. The method of embodiment N42, wherein the subject was previously treated for cancer.
   49. The method of embodiment N42, wherein the subject was previously in remission.
   50. The method of any of embodiments N1-N49, wherein the imaging agent is selected from a radionuclide, a positron-emitting isotope, a fluorophores, antibodies, a bioluminescent molecule, a chemiluminescent molecule, a photoactive molecule, a metal, an electron-dense reagent, an enzyme, a magnetic contrast agent, a quantum dot, a nanoparticles, biotin, digoxigenin, a hapten, or a protein or other entity which can be made detectable.
   51. A method of treating cancer in a subject in need, comprising administering the method of any one of embodiments N1-N50 and administering an anti-cancer therapy that does not comprise microbubbles.
   52. The method of embodiment N51, wherein the anti-cancer therapy that does not comprise microbubbles is chemotherapy, hormonal therapy, radiotherapy, or immunotherapy.
   53. The method of embodiment N51, wherein the anti-cancer therapy that does not comprise microbubbles is an anti-cancer agent that does not comprise microbubbles.
   54. The method of embodiment N53, wherein the anti-cancer agent that does not comprise microbubbles is selected from an alkylating agent, an antimetabolite, a natural product, a chemotherapeutic, a hormone, polypeptide, or a small molecule having utility in methods of treating cancer.
   55. The method of embodiment N54, wherein the anti-cancer agent that does not comprise microbubbles is gemcitabine.
   56. The method of embodiment N54, wherein the therapeutic agent that does not comprise microbubbles is temozolomide.
   57. The method of any of embodiments N51-N56, wherein the active agent that does not comprise microbubbles further comprises a pharmaceutically acceptable excipient.
   58. A kit for use in the treatment of a target tissue with an active agent, the kit comprising a first and second microbubble composition, wherein (i) the active agent is a therapeutic agent or an imaging agent (ii) the first microbubble composition comprises first microbubbles and does not comprise the active agent, and (iii) the second microbubble composition comprises second microbubbles complexed with the active agent.
   59. The kit of embodiment N58, wherein the first microbubble composition, the second microbubble composition, or both are formulated for intravenous administration.
   60. The kit of embodiment N58 or N59, wherein the first and/or second microbubbles have a mean or median diameter of about 1 micron to about 5 microns, or about 2.5 microns to about 4 microns.
   61. The kit of any one of embodiments N58-N60, wherein the first and/or second microbubbles comprise a targeting moiety.
   62. The kit of embodiment N61, wherein the targeting moiety is a molecule selected from an antibody, antibody fragment, a binding protein, a binding protein fragment, a receptor, a receptor fragment, a receptor ligand, a peptide, a polypeptide, a polynucleic acid, a polysaccharide, a lipid, a polymer, tumor associated antigen, tissue specific antigen, or vascular associated antigen, or any combination of molecules thereof.
   63. The kit of embodiment N62, wherein the targeting moiety comprises (a) a VEGF polypeptide or single-chain variant thereof, (b) a VCAM1 antibody or epitope-binding fragment thereof, or (c) a PSMA antibody or epitope-binding fragment thereof.
   64. The kit of any one of embodiments N58-N63, wherein the active agent comprises a protein or a nucleic acid, optionally wherein the nucleic acid comprises an shRNA, an siRNA, an miRNA, an mRNA, RNA, a vector, a plasmid, DNA, or any combination thereof.
   65. The kit of any one of embodiments N58-N63, wherein the active agent comprises an anti-cancer agent.
   66. The kit of any one of embodiments N58-N63, wherein the active agent comprises a virus.
   67. The kit of embodiment N66, wherein the virus is a tropism modified adenovirus.
   68. The kit of embodiment N66 or N67, wherein replication of the virus is under control of a cancer-selective promoter.
   69. The kit of any one of embodiments N66-N68, wherein the virus comprises a polynucleotide encoding an shRNA, an siRNA, or an antisense RNA.
   70. The kit of any one of embodiments N66-N68, wherein the virus comprises a polynucleotide encoding an MDA-7/IL-24 protein.
   71. The kit of any one of embodiments N58-N63, wherein the active agent comprises an MDA-7/IL-24 protein or a polynucleotide encoding the MDA-7/IL-24 protein.
   72. The kit of embodiment N70 or N71, wherein the MDA-7/IL-24 protein is a fusion protein.
   73. The kit of any one of embodiments N70-N72, wherein the MDA-7/IL-24 protein comprises an insulin signal peptide.
   74. The kit of any one of embodiments N70-N73, wherein the MDA-7/IL-24 protein comprises an amino acid sequence that is at least 90% identical to SEQ ID NO: 3 or 4.
   75. The kit of any one of embodiments N70-N73, wherein the MDA-7/IL-24 protein comprises a mutation corresponding to (a) a change of K122R relative to SEQ ID NO: 2, (b) a change of K73R relative to SEQ ID NO: 3, (c) a change of K19R relative to SEQ ID NO: 4, or (d) SEQ ID NO: 18.
   76. The kit of any one of embodiments N58-N63, wherein the active agent comprises an MDA-9/Syntenin polynucleotide inhibitor.
   77. The kit of any one of embodiments N66-N68, wherein the virus comprises a polynucleotide encoding an MDA-9/Syntenin inhibitor.
   78. The kit of embodiment N76 or N77, wherein the MDA-9/Syntenin polynucleotide sequence comprises a sequence that encodes an siRNA, an shRNA, a miRNA, lncRNA, or antisense RNA sequence.
   79. The kit of embodiment N78, wherein the sequence comprises SEQ ID NO: 20.
   80. The kit for use of any one of embodiments N58-N79, wherein the use comprises: (a) administration of the first microbubble composition, (b) a first ultrasound administration directed to the target tissue that disrupts the first microbubbles, (c) administration of the second microbubble composition after the first ultrasound administration, and (d) a second ultrasound administration directed to the target tissue that disrupts the second microbubbles.
   81. The kit for use of embodiment N80, wherein administration of the second microbubble composition is within about 60 minutes, 30 minutes, 10 minutes, or 5 minutes of administration of the first microbubble composition.
   82. The kit for use of embodiment N80 or N81, wherein the target tissue comprises a tumor.
   83. The kit for use of embodiment N82, wherein the tumor is a metastatic tumor.
   84. The kit for use of embodiment N82 or N83, wherein the tumor is located in the brain, a breast, a lung, the gastrointestinal system, a bone, the peritoneal cavity, pancreas, intestine, skin, head, neck, oral cavity, spinal cord, or liver of the subject.
   85. The kit for use of embodiment N82 or N83, wherein the tumor comprises glioblastoma, melanoma, breast cancer, pancreatic cancer, liver cancer, prostate cancer, colon cancer, oral cancer, head and neck cancer, spinal cord cancer, neuroblastoma, kidney cancer, or lung cancer.
   86. The kit for use of embodiment N80, wherein the target tissue is located within the brain, pancreas, stomach, intestines, bones, skin, oral cavity, the peritoneal cavity, spinal cord, head, neck, kidney, or liver of the subject.
   87. The kit for use of any one of embodiments N82-N86, wherein the target tissue is in the brain of the subject.

s88. The kit for use of any one of embodiments N82-N86, wherein the target tissue is in the pancreas of the subject.

89. The kit for use of any one of embodiments N80-N88, wherein administration of the first microbubble composition is in an amount effective to increase delivery of the active agent to the target tissue.
90. The kit for use of embodiment N89, wherein administration of the first microbubble composition is in an amount effective to increase delivery of the active agent across the blood-brain barrier.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entireties for all purposes.

### Example 1: Double Treatment with MBs (FUS-DMB Delivery) Approach

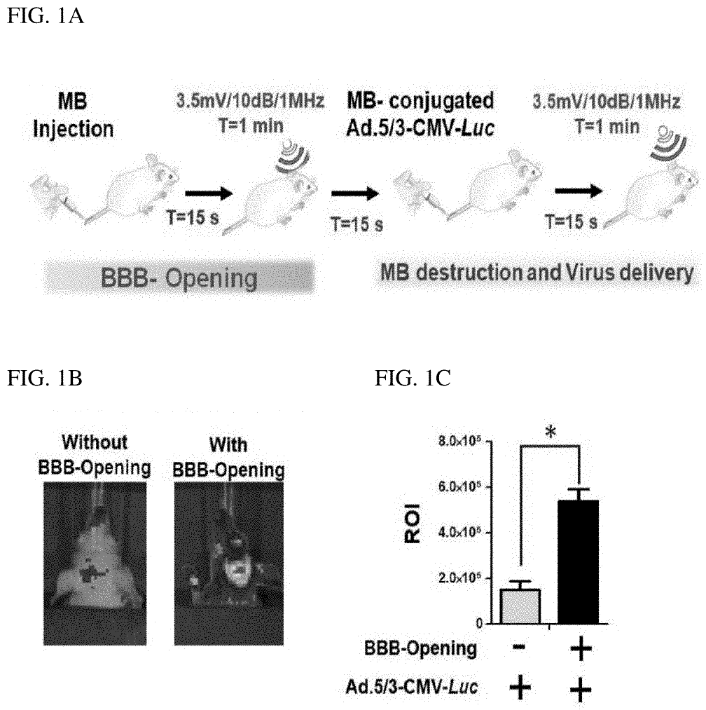

We used FUS with naked MBs (microbubbles lacking an active agent) to transiently open the BBB to permit access of adenoviruses expressing luciferase (delivered subsequently in MBs and released by FUS using UTMD). As shown in FIGS. 1A-1C, FUS (with UTMD) temporarily opens the BBB and allows the adenovirus expressing luciferase (Ad.CMV-Luc incorporated in MBs) to enter and then to be released in the brain following a second FUS. This protocol did not cause any overt toxic effects, since mice survived at least 3 weeks after the procedure without any noticeable symptoms.

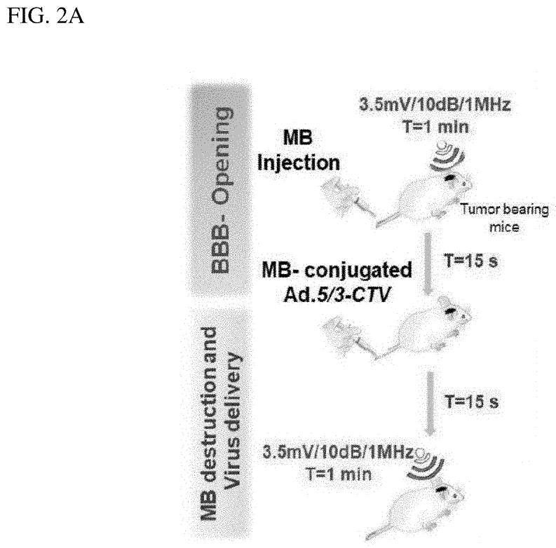

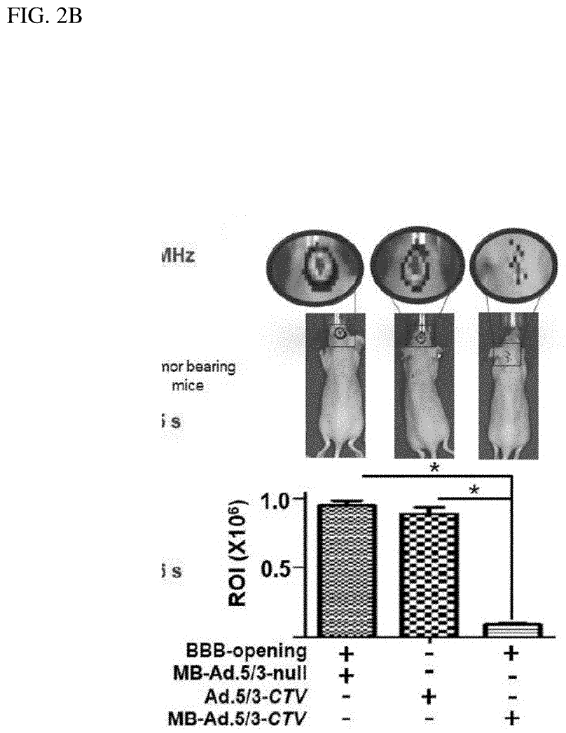

We next tested the potential of targeted delivery of therapeutic adenoviruses (Ad.5/3-CTV) using FUS and UTMD to brain tumors using a focused ultrasound (FUS) dual MB (FUS-DMB) delivery approach. GBM-6, a highly aggressive primary human glioblastoma cell line that recapitulates the human disease (very invasive in nature with fingerlike projections of tumor cells in the brain) when injected intracranially in mouse brain were used in this study. We systemically delivered MB-encapsulated Ad.5/3-CTV in GBM-6-Luc (GBM-6 clone expressing luciferase) to brain tumor-bearing mice and monitored their therapeutic response through IVIS. The experimental group that received FUS with empty MBs and UTMD prior to therapeutic delivery of MB-Ad.5/3-CTV by FUS and UTMD the focused ultrasound (FUS) dual MB (FUS-DMB) delivery strategy demonstrated robust tumor suppression as indicated by decreased fluorescence and enhanced animal survival (FIGS. 2A and 2B).

These findings document that therapeutic adenoviruses (Ad.5/3-CTV) can be administered in a “stealth manner” in the circulation and delivered to target GBM tumors in the brain by means of MBs+FUS after opening the BBB with naked MBs and FUS. This strategy resulted in significant therapeutic activity without the need for surgery in animals with GBM-6 tumors. In the context of patients with GBM, this strategy would allow administering of therapeutic viruses in MBs systemically and application of FUS in patients following opening up of the BBB with naked MBs and FUS using a FUS-DMB delivery approach without the need for surgical debulking of the tumor (see, e.g., FIG. 2B). In principle, this approach could be used for primary GBM and recurrent GBM (which occurs in most patients after primary tumor removal and chemotherapy and/or radiation therapy), without the need for surgery. This approach can also be applied to treat metastatic tumors (from other sites including the breast or melanoma) that colonize in the brain.

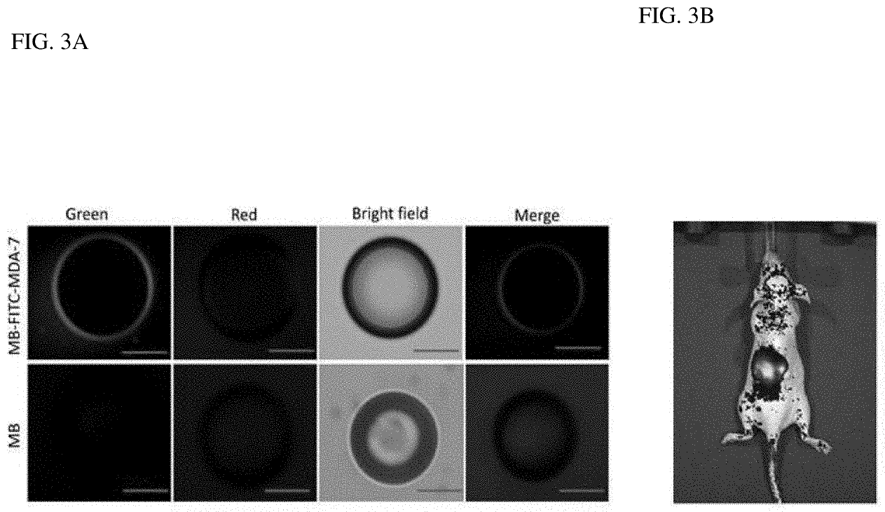

In addition to delivering therapeutic viruses systemically, we also tested the possibility of using MBs and a UTMD approach to deliver therapeutic proteins in vivo. His tagged MDA-7/IL-24 (His-MDA-7) was purified by using Ni-NTA column chromatography. 1 mg/ml of His-MDA-7 solution was mixed with Alexa Fluor 488 containing 0.1 M Sodium bicarbonate, followed by removal of unincorporated dye by centrifugation using centricon (3 kD cut off MW). Confirmation of labeling of His-MDA-7 (Alexa Fluor-His-MDA-7) was obtained spectroflurometrically based on emission spectra changes. The Alexa Fluor-His-MDA-7 was incubated overnight with MBs and localization of the labeled protein complex in the MBs was monitored by green fluorescence and found to be associated with the lipid shell of the MB (FIG. 3A). To test delivery of labeled Alexa Fluor-His-MDA-7 complexed with MBs in vivo using the UTMD approach, DU-145 tumor xenografts were established in the left flank of nude mice. The Alexa Fluor-His-MDA-7 compexed with MBs was administered in the tail vein of the mice. As shown in FIG. 3B, UTMD Alexa Fluor-MDA-7 was located predominantly in the tumor in the left flank of mice. These studies confirm the utility of MB s to deliver therapeutic proteins in a target-specific manner in vivo when administered intravenously and then subjected to ultrasound.

### Example 2: Enhancing Delivery of MBs to Tumors, Metastases and the Tumor Vasculature

A variety of molecular markers are overexpressed on the vascular endothelium in the tumor vasculature. To facilitate targeting of MBs, MBs can be decorated with a targeting moiety (also referred to as a targeting ligand), such as an antibody. A targeting moiety can be attached to the surface of MBs via biotin-streptavidin spacer, or via a direct chemical coupling, preferably, oriented coupling via thiol-maleimide. Of direct therapeutic relevance, the latter approach provides a successful strategy for clinical translation.

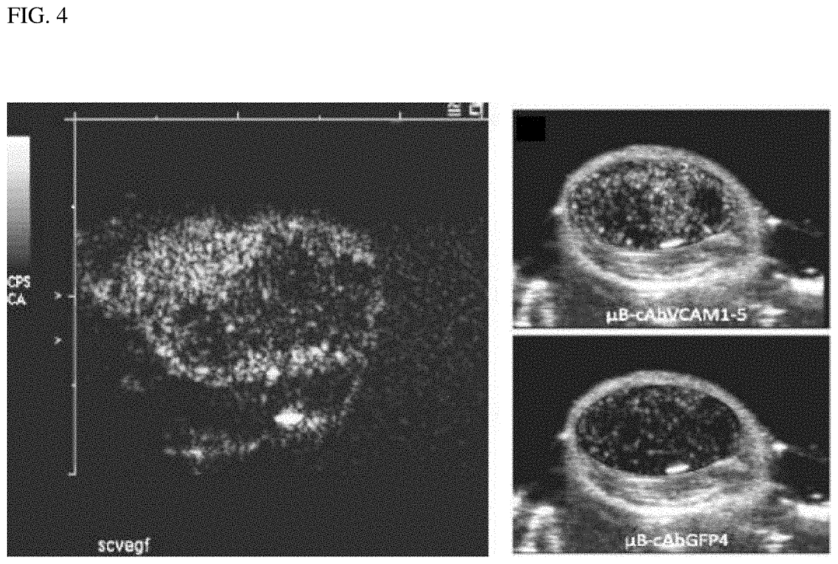

We achieved successful MB targeting to VEGFR2 by attaching a single- chain VEGF molecule to the microbubble surface. Under ˜100,000 molecules of targeting ligand per bubble is sufficient to ensure successful targeting to the tumor vasculature that overexpresses VEGFR2 (FIG. 4, left panel). Another target for MB targeting to tumor vasculature is VCAM-1. We have successfully placed anti-VCAM-1 antibody fragments on the MB surface and achieved targeted imaging in a murine tumor animal model in vivo (FIG. 4, right panel).

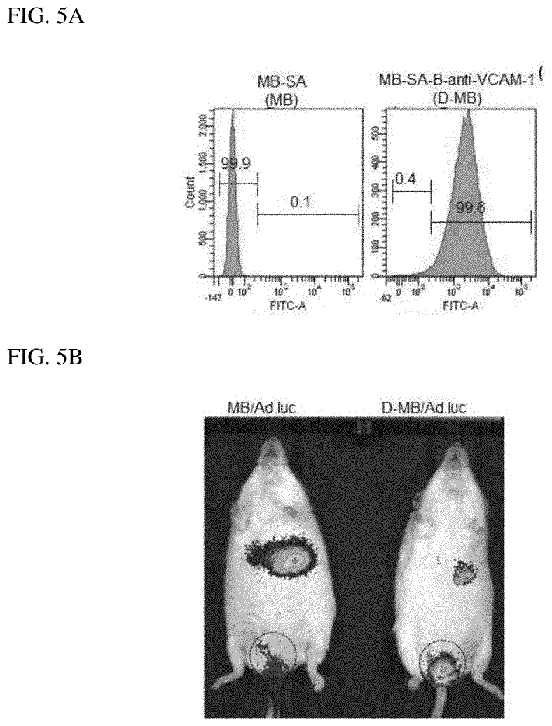

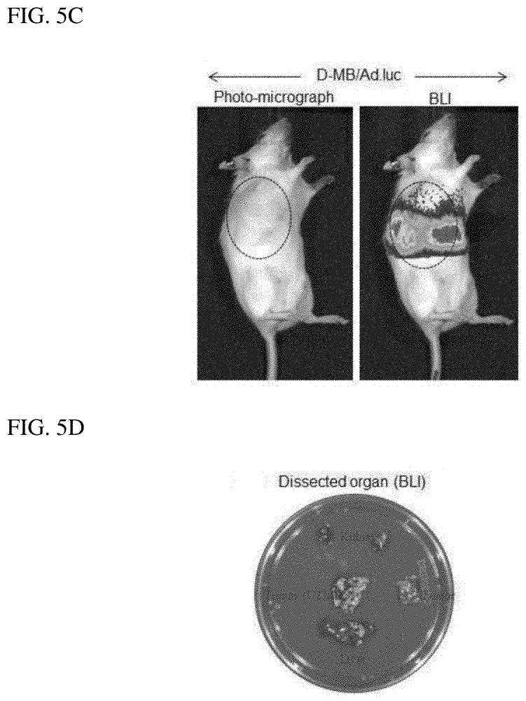

In-order to make a targeted MB, it was functionalized with mouse anti-V-CAM-1 antibody using biotin-streptavidin conjugation chemistry as confirmed by Flow-cytometric data (FIG. 5A). Targeted or Decorated MB (D-MB) was further validated for site-specific delivery in two transgenic animals: Hi-Myc (Prostate cancer) (FIG. 5B) and PyMT (Breast Cancer) (FIG. 5C). MB complexed Ad.luc was administrated through the I.V. route and followed by sonoparation at the corresponding tumor sites. BLI imaging was done after 72 h of post delivery using IVIS Spectrum (FIG. 5B and FIG. 5C). In both models, it was shown that Ad.luc was delivered at the site of sonoporation. Trace BLI signal was obtained from liver. Improved delivery may be achieved by using a next generation sonoporation apparatus that can provide a more directed FUS. Other than liver there was hardly any traces of Ad.luc delivery obtained in secondary tumor (non-sonoporated mammary tumors in PyMT model, FIG. 5D), or kidney indicating the utility of the targeted UTMD approach for systemic delivery of Ad and thus in principle providing an effective means of delivery of therapeutic virus systemically with enhanced payload delivery.

This approach can be used to facilitate delivery of therapeutic viruses, recombinant proteins and chemotherapy to GBM or metastatic tumors in the brain (using our double MB UTMD approach) as well as primary tumors, metastases and the tumor vasculature in different anatomic sites in the body.

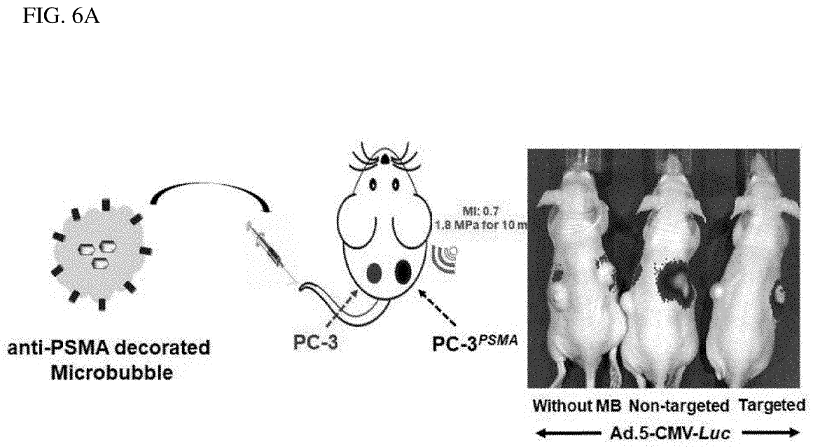

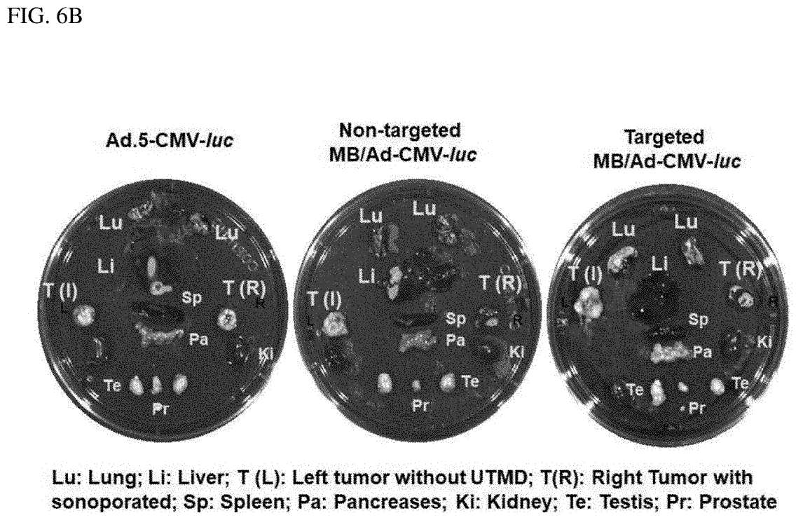

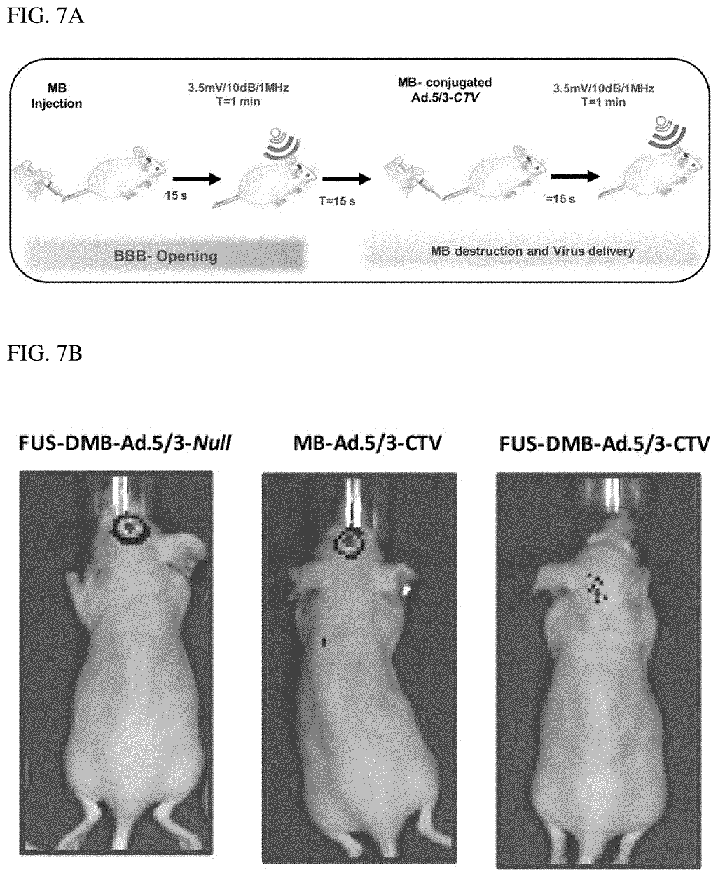

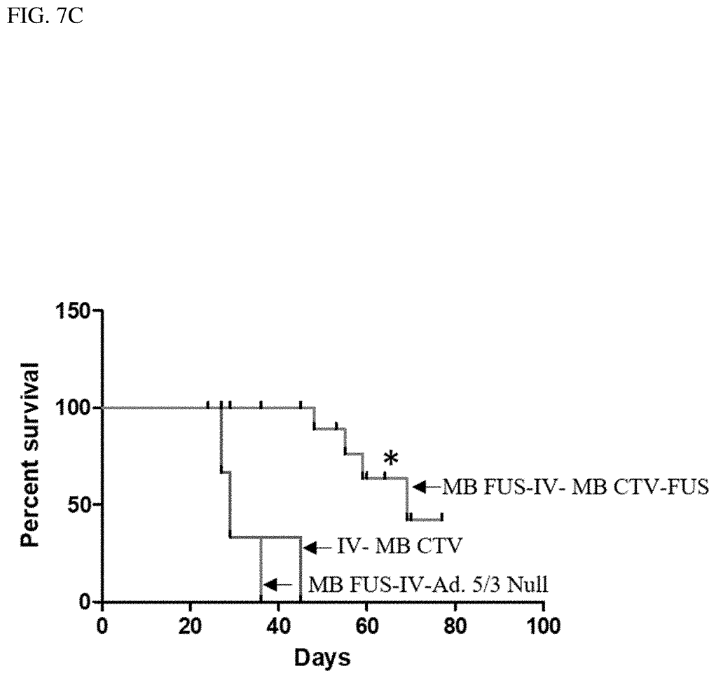

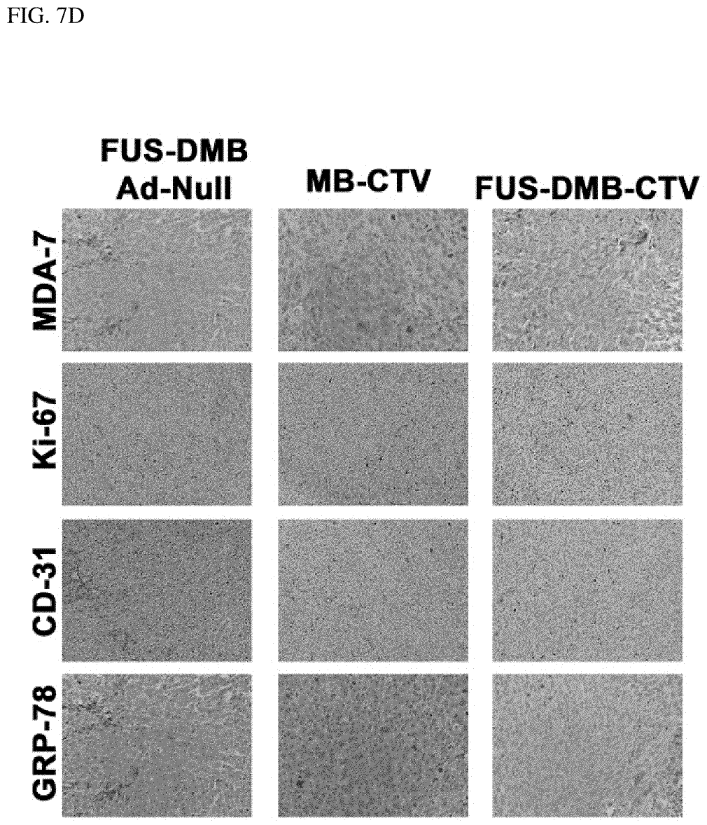

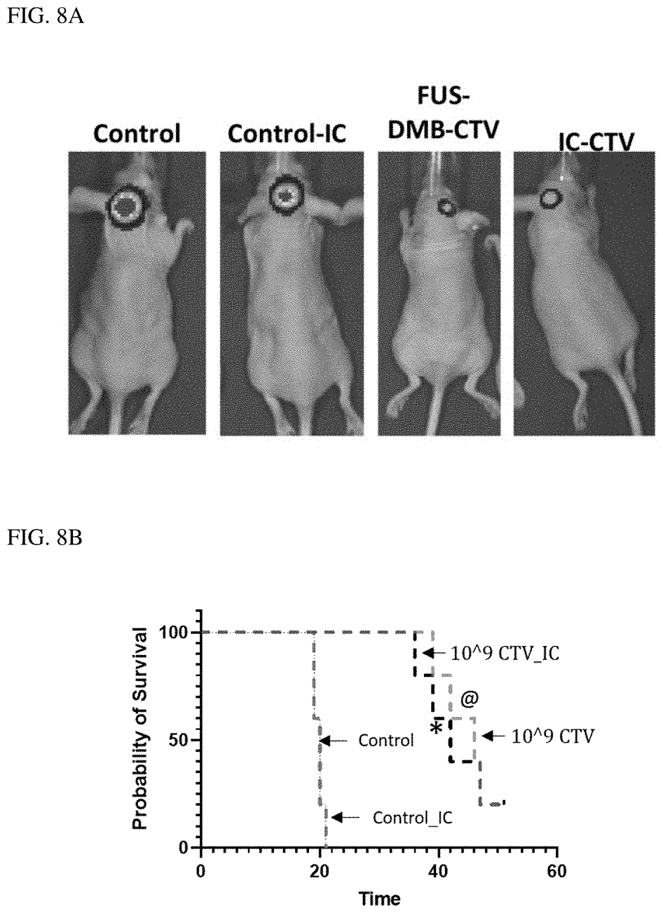

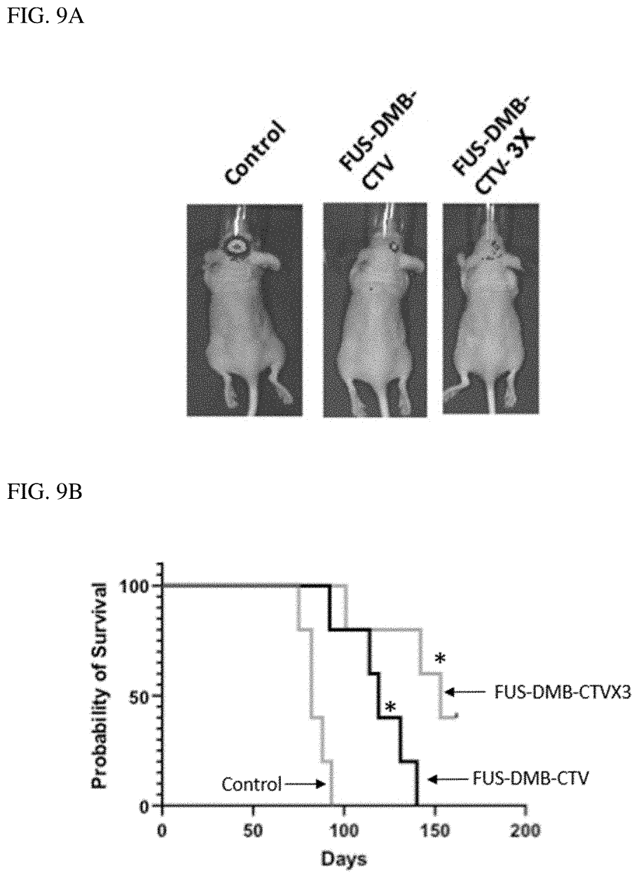

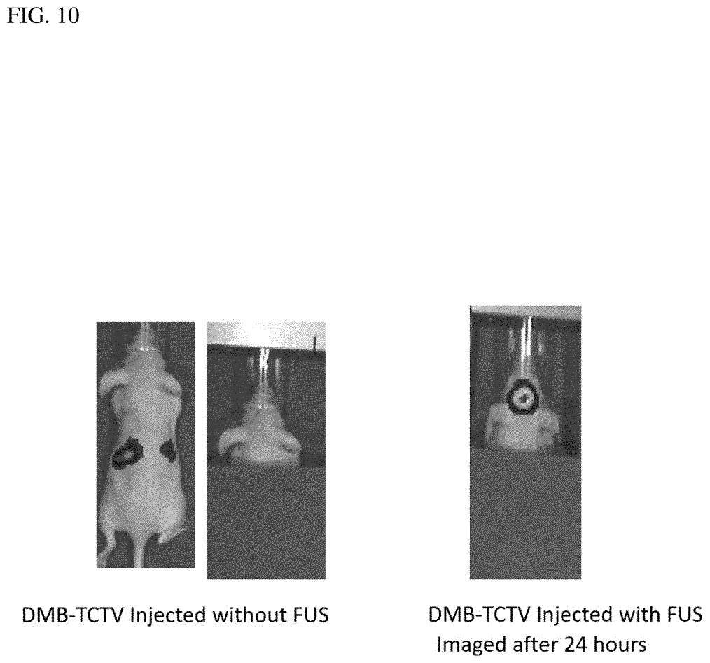

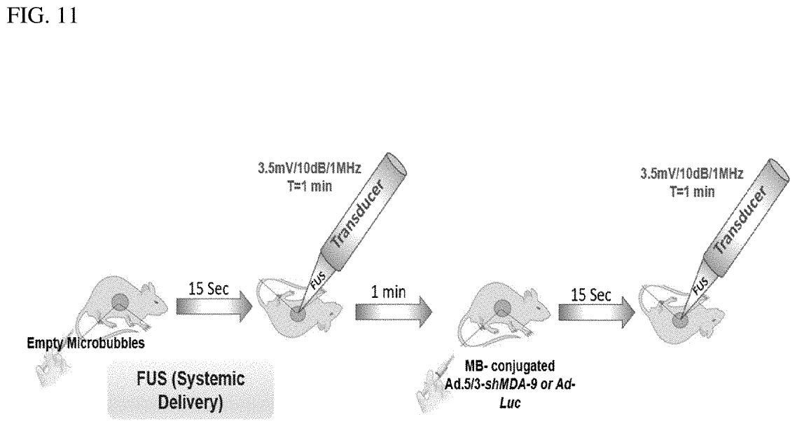

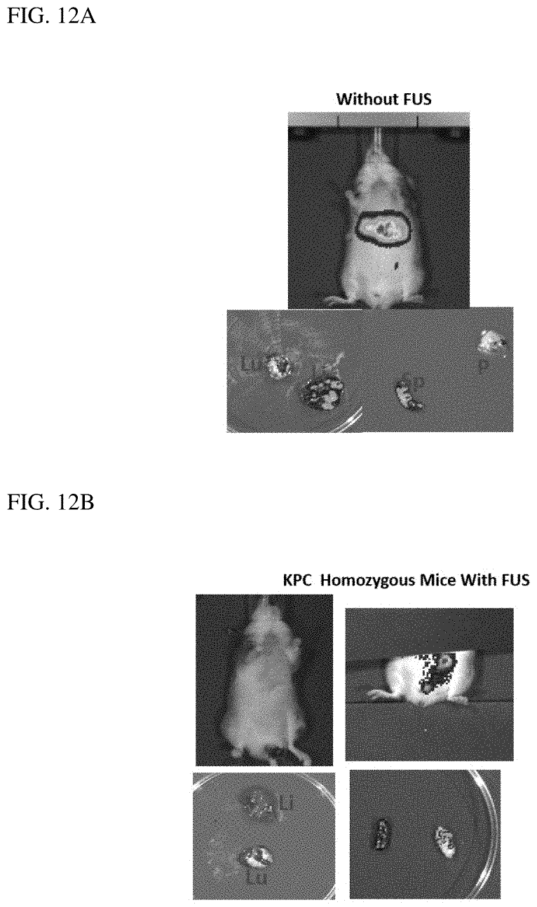

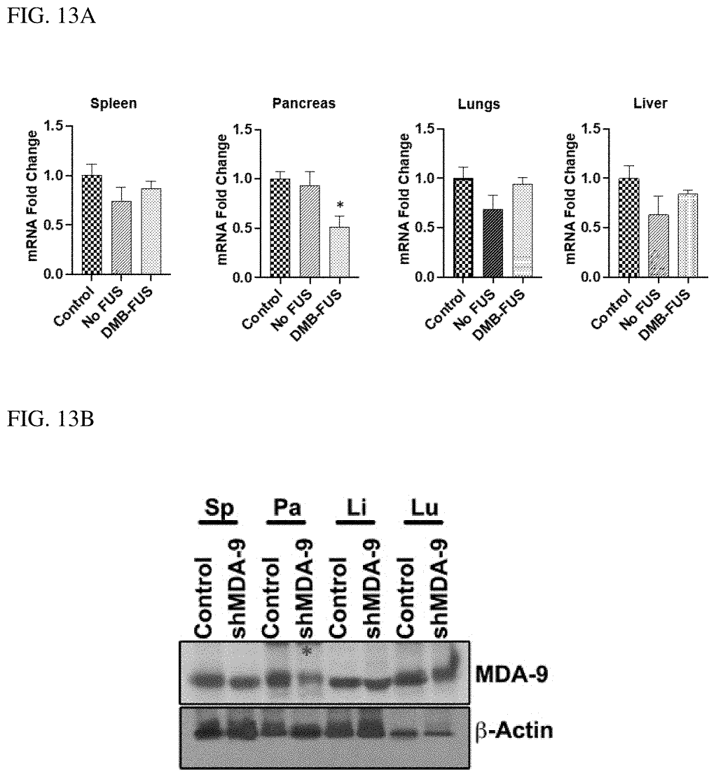

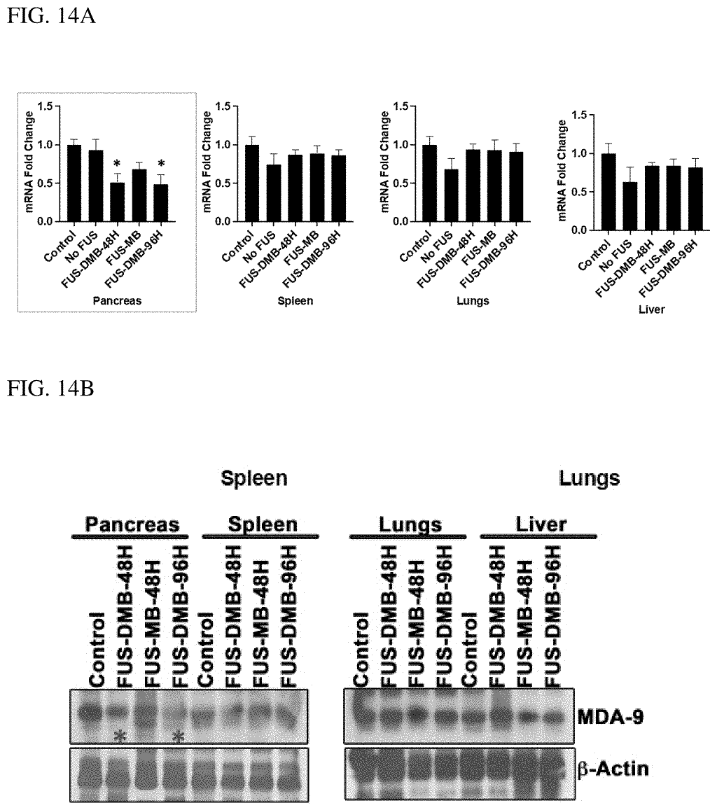

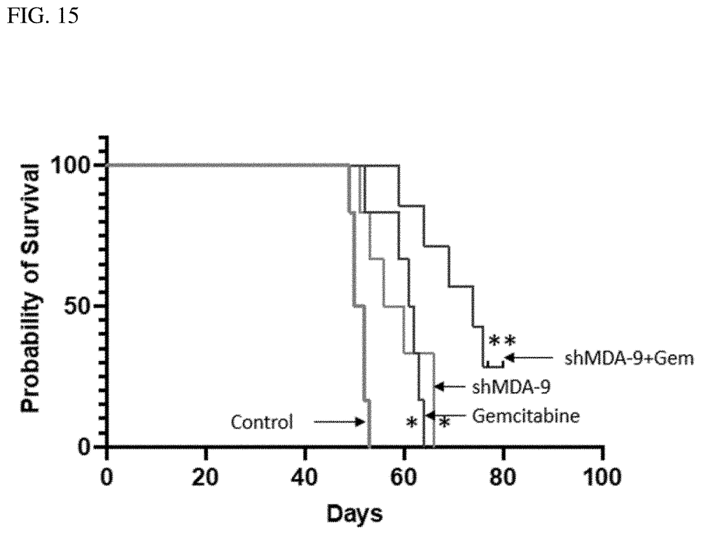

We have generated a targeted (decorated) MB (d-MB) that binds specifically with prostate specific membrane antigen (PSMA). The targeted (decorated MBs) called PE-anti-PSMA-MB binds specifically with PC-3-PIP cells (PC-3 cell overexpressing PSMA) (FIGS. 6A and 6B), whereas the binding of non-targeted (non-decorated) MBs (PE-IgG-MB) showed limited binding in comparison with the decorated MBs (data not shown). Next, we investigated the effectiveness of delivery of d-MBs in an in vivo nude mouse model containing PC-3 and PC-3-PIP tumor xenografts. In order to observe the site-specific delivery of gene or therapeutic Ads by MBs coupled with the UTMD technology, PC-3 and PC-3-PIP cells were injected s.c. into the left and right flank, respectively, of nude mice (FIGS. 6A and 6B). There was no difference in tumor growth in both flanks, and the mice were injected with MBs by tail-vein injection after the tumor size of both flanks reached ˜100 mm³. Ad.5/3-CMV-luc conjugated with MBs (Simple MB) and Ad.5/3-CMV-luc conjugated to anti-PSMA-MBs (Targeted MBs) and the free Ad.5/3-CMV-luc were injected, the mice were sonoporated in the right flank (PC-3-PIP bearing tumor) using the UTMD approach, and the mice were imaged 72-h post-injection of Ad.5/3-CMV-luc delivery. It was found that both simple MBs and targeted MBs could deliver Ads in the targeted site of sonoporation. In the case of simple MBs, the delivery of Ads was simply due to its release from MBs in the region where ultrasound was applied, and the signal strength of BLI was lower as compared to targeted MBs. Moreover, the signal in the case of the simple MBs was in a wider area compared to the focused release of Ads by the targeted MBs, indicating more specific delivery of Ads by targeted MBs. The more specific delivery of Ads by targeted MBs might be due to active binding of targeted MBs on the surface of tumor cells followed by the release of Ads upon application of ultrasound at the target site. Thus, the use of targeted MBs restricted nonspecific delivery of Ads in the surrounding tumor region. Delivery effects may be further enhanced through combination of targeted MBs with a dual-MB approach, as described herein.

### Example 3: Focused Ultrasound (FUS) Dual MB (FUS-DMB) Delivery of Ad.5/3-CTV Significantly Prolongs the Survival of Human GBM Tumor Bearing Mice

### Intracranial Injections of GBM6 Cells:

The mice were anesthetized via i.p. administration of (ketamine, 40 mg/kg; xylazine, 3 mg/kg) and immobilized in a stereotactic frame. A 24-gauge needle attached to a Hamilton syringe was inserted into the right basal ganglia to a depth of 3.5-mm and then withdrawn 0.5-mm to make space for tumor cell accumulation. The entry point at the skull was 2-mm lateral and 1-mm dorsal to the bregma. Intracerebral injection of 30,000 glioma cells (GBM6/GBM6-Luc/GSC-8-11-Luc) in 5 μ1 of DMEM medium was performed over 10 minutes. The skull opening was enclosed with sterile bone wax, and the skin incision was closed using sterile surgical staples.

### Ad.5/3-CTV Treatment Procedure:

Adenoviral vectors were administered 10 days after tumor cell implantation via stereotactic injection into the intracerebral tumor using the same anesthesia procedure and stereotactic frame coordinates described above. Viral vectors suspended in 2 μl of phosphate-buffered saline (PBS) were delivered by slow infusion over a 6-minute period. These mice were then imaged every week until the IACUC end point and used for survival analysis.

### Microbubble-Adenovirus Complex:

Perfluorocarbon MBs were reconstituted in 1 ml of PBS containing 1×10¹¹ viral particles of the indicated Adenovirus and incubated at 4° C. for 2 hours. After the incubation, unenclosed surface-associated Ads were inactivated by treating with 20% FBS for 3 h at 4° C. and washed twice to remove unbound adenovirus. Finally, MB/Ad was dissolved in lml of PBS prior to treatment. Complement treated MB s/Ads were systemically injected via tail vein and sonoporated (using focused ultrasound).

Generation of Focused Ultrasound (FUS) Waves and Treatment of Mice to Cross Blood Brain Barrier (BBB):

Focused ultrasound (FUS) utilizes the same concept of acoustic wave propagation as the more widely known diagnostic ultrasound applications. However, instead of acquiring and displaying echoes generated at several tissue interfaces for imaging, FUS employs concave transducers that usually have either a single geometric focus or use phased arrays to electronically steer it, at which most of the power is delivered during sonication in order to induce mechanical effects, thermal effects, or both. The FUS transducer (2.25 MHz, 0.50 in. Element Diameter, Standard Case Style, Straight UHF Connector, purchased from Olympus America Inc.) is used to perform sonication immediately following bubble administration (15 seconds). The transducer is driven by a function generator (AGI-E4436B, Agilent Technologies, Palo Alto, Calif., USA) through a power amplifier (E&I 3100LA, ENI Inc., Rochester, N.Y., USA). A cone filled with degassed and distilled water is attached to the transducer system. Mice were injected with 100 μl of diluted microbubbles and immediately after IV FUS was applied (3.5 mV, 10 dB, 1 MHz), BBB opening was observed as per Evans blue experiment.

### Example 4: Systemic Administration of Ads using FUS-DMB

### FUS-DMB Systemic Delivery:

100 μl of diluted MB was injected through the tail vein and allowed to circulate for 15 sec. After 15 sec mice were sonicated (ultrasound) for 1 minute as described herein. Next the mice were injected I/V with 100 μl microbubble containing Ads and sonicated for 1 minute after allowing the bubbles to circulate for 15 sec. These animals were imaged using IVIS imager and followed for survival analysis.

### Example 5: Systemic Administration of Ads using FUS-DMB to Target the Pancreas

Pancreatic Delivery of shMDA-9 in KPC Mouse Model:

The KPC mouse model of pancreatic ductal adenocarcinoma (PDAC) was first described in 2005 and incorporates, through Cre-Lox technology, the conditional activation of mutant endogenous alleles of the Kras and Trp53 genes. Specifically, an activating point mutation (G12D) in Kras and a dominant negative mutation in Trp53 (R172H) are conditionally activated in the mouse pancreas by breeding LSL-KrasG12D/+; LSL-Trp53R172H/+ mice to Pdx-1 -Cre mice that express Cre recombinase under the expression of the pancreas-specific Pdx-1 promoter. Cre-mediated recombination acts to excise the loxP-flanked stop codon (LSL), an event that occurs only in cells expressing Cre, thereby leading to conditional expression of mutant Kras and Trp53 genes specifically in the mouse pancreas.

### Intravenous FUS-DMB:

100 μl of diluted MB was injected through the tail vein and allowed to circulate for 15 sec. After 15 sec mice were sonicated (ultrasound) for 1 minute as described above in the pancreas region. Next the mice were injected I/V with 100 μl microbubble containing Ads (Luc/shMDA-9) and sonicated for 1 minute after allowing the bubbles to circulate for 15 sec. These animals were imaged using IVIS imager and followed for survival analysis.

### Western Blot Analysis:

First, we injected empty microbubbles and applied FUS for 1 minute using above parameters and a minute after complement treated MBs/Ads were systemically injected via tail vein (100 μl) in KPC homozygous mice and sonoporated using FUS. These mice were observed for indicated time points and either imaged or euthanized and different organs were collected (spleen, pancreas, lungs and liver). These organs were lysed and proteins were isolated using standard protocols and equal amounts of protein were resolved using SDS-PAGE. Western blot analysis was performed using MDA-9 specific antibody. β-Actin was used as loading control.

### Real-time PCR Analysis:

First, we injected empty microbubbles and applied FUS for 1 minute using above parameters and a minute after complement treated MBs/Ads were systemically injected via tail vein (100 μl) in KPC homozygous mice and sonoporated using FUS. These mice were observed for indicated time points and either imaged or euthanized and different organs collected (spleen, pancreas, lungs and liver). These organs were lysed, and RNA was isolated using a standard protocol and equal amounts of RNA were used to synthesize cDNA according to the manufacturer's protocol. Real-time PCR was performed to check MDA-9 mRNA levels. Mouse GAPDH was used as transcription control.

### Statistical Analysis

All data represent mean±S.D. from three independent experiments. Statistical analysis was performed using either Student t test (Microsoft excel), Pearson Correlation (GraphPad prism software). P<0.05 was considered significant.

### Survival Analysis:

The Kaplan Meier Curve was generated using PRISM Graph PAD software and it is used to estimate the survival function. Gemcitabine was given at a dose of 20 mg/kg via intraperitoneal injections.

### REFERENCES

Lawrie A, et al. (2000) Microbubble-enhanced ultrasound for vascular gene delivery. Gene Ther 7(23):2023-2027.

Ng K Y & Liu Y (2002) Therapeutic ultrasound: its application in drug delivery. Med Res Rev 22(2):204-223.

Larina I V, et al. (2005) Enhancement of drug delivery in tumors by using interaction of nanoparticles with ultrasound radiation. Technol Cancer Res Treat 4(2):217-226.

Howard C M, et al. (2006) Ultrasound guided site specific gene delivery system using adenoviral vectors and commercial ultrasound contrast agents. J Cell Physiol 209(2):413-421.

Goldberg B B, Liu J B, & Forsberg F (1994) Ultrasound contrast agents: a review. Ultrasound Med Biol 20(4):319-333.

Pitt W G, Husseini G A, & Staples B J (2004) Ultrasonic drug delivery—a general review. Expert Opin Drug Deliv 1(1):37-56.

Ignee A, Straub B, Schuessler G, & Dietrich C F (2010) Contrast enhanced ultrasound of renal masses. World J Radiol 2(1):15-31.

Ignee A, et al. (2010) The value of contrast enhanced ultrasound (CEUS) in the characterisation of patients with renal masses. Clin Hemorheol Microcirc 46(4):275-290.

Lindner J R, et al. (2001) Ultrasound assessment of inflammation and renal tissue injury with microbubbles targeted to P-selectin. Circulation 104(17):2107-2112.

Greco A, et al. (2010) Eradication of Therapy-resistant Human Prostate Tumors Using an Ultrasound-guided Site-specific Cancer Terminator Virus Delivery Approach. Molecular Therapy 18(2):295-306.

Dash R, et al. (2011) Apogossypol derivative BI-97C1 (Sabutoclax) targeting Mcl-1 sensitizes prostate cancer cells to mda-7/IL-24-mediated toxicity. Proc. Natl. Acad. Sci. U.S.A. 108(21):8785-8790.

Kegelman, T. P., et al. (2014) MDA-9/Syntenin is a Key Regulator of Glioma Pathogenesis. Neuro-Oncolgy 16(1):50-61.

Bhoopathi, P., et al. (2021) Theranostic Tripartite Cancer Terminator Virus for Cancer Therapy and Imaging. Cancer 13(4):857.

1.-90. (canceled)

91. A method of administering an active agent to a target tissue, wherein the active agent is a therapeutic agent or an imaging agent, the method comprising:
(a) administering to a subject a first microbubble composition, the first microbubble composition comprising first microbubbles and not comprising the active agent;
(b) a first ultrasound administration directed to the target tissue that disrupts the first microbubbles;
(c) administering to the subject a second microbubble composition after the first ultrasound administration, the second microbubble composition comprising second microbubbles complexed with the active agent; and
(d) a second ultrasound administration directed to the target tissue that disrupts the second microbubbles and releases the active agent to the target tissue.

92. The method of claim 91, wherein the second microbubble composition is administered within about 60 minutes of administering the first microbubble composition.

93. The method of claim 91, wherein the first and/or second microbubbles comprise a targeting moiety.

94. The method of claim 93, wherein the targeting moiety is a molecule selected from an antibody, antibody fragment, a binding protein, a binding protein fragment, a receptor, a receptor fragment, a receptor ligand, a peptide, a polypeptide, a polynucleic acid, a polysaccharide, a lipid, a polymer, a tumor associated antigen, a tissue type-associated antigen, a vascular associated antigen or any combination of molecules thereof.

95. The method of claim 91, wherein the target tissue comprises a tumor.

96. The method of claim 95, wherein the tumor is located in the brain, a breast, a lung, the gastrointestinal system, a bone, the peritoneal cavity, the oral cavity, pancreas, intestine, skin, head, neck, spinal cord, or liver of the subject.

97. The method of claim 91, wherein the active agent comprises a protein or a nucleic acid, optionally wherein the nucleic acid comprises an shRNA, an siRNA, an miRNA, an lncRNA, an mRNA, RNA, a vector, a plasmid, DNA, or any combination thereof.

98. The method of claim 91, wherein the active agent comprises an anti-cancer agent.

99. The method of claim 91, wherein the active agent comprises a virus.

100. The method of claim 97, wherein the active agent comprises an MDA-7/IL-24 protein, said MDA-7/IL-24 protein further comprising an insulin signal peptide, or a polynucleotide encoding an MDA-7/IL-24 protein further comprising an insulin signal peptide.

101. The method of claim 100, wherein the MDA-7/IL-24 protein comprises a mutation corresponding to (a) a change of K122R relative to SEQ ID NO: 2 (b) a change of K73R relative to SEQ ID NO: 3, (c) a change of K19R relative to SEQ ID NO: 4, or (d) SEQ ID NO: 18.

102. The method of claim 91, wherein the active agent comprises an MDA-9/Syntenin inhibitor.

103. The method of claim 91, further comprising:
(e) administering to the subject a third microbubble composition after the second ultrasound administration, the third microbubble composition comprising second microbubbles complexed with a second active agent or imaging agent; and
(f) a third ultrasound administration directed to the target tissue that disrupts the third microbubbles and releases the second active agent or imaging agent to the target tissue.

104. The method of claim 91, wherein the microbubble composition comprises two or more active agents.

105. The method of claim 91, wherein the subject has cancer or is at risk of having cancer.

106. The method of claim 95, wherein the cancer is a solid tumor cancer.

107. The method of claim 91, wherein the imaging agent is selected from a radionuclide, a positron-emitting isotope, a fluorophores, antibodies, a bioluminescent molecule, a chemiluminescent molecule, a photoactive molecule, a metal, an electron-dense reagent, an enzyme, a magnetic contrast agent, a quantum dot, a nanoparticles, biotin, digoxigenin, a hapten, or a protein or other entity which can be made detectable.

108. A method of treating cancer in a subject in need, comprising administering the method of claim 91 and administering an anti-cancer therapy that does not comprise microbubbles.

109. The method of claim 108, wherein the anti-cancer therapy that does not comprise microbubbles is chemotherapy, hormonal therapy, radiotherapy, or immunotherapy.

110. A kit for use in the treatment of a target tissue with an active agent, the kit comprising a first and second microbubble composition, wherein (i) the active agent is a therapeutic agent or an imaging agent (ii) the first microbubble composition comprises first microbubbles and does not comprise the active agent, and (iii) the second microbubble composition comprises second microbubbles complexed with the active agent.


SEQUENCE LISTING (20 sequences)

SEQ ID NO: 1 - DNA, 621 bp, Artificial sequence
  Synthetic polynucleotide
  misc_feature (1)..(621)
atgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggttcctgctattccggagagcatttaaacagttggacgtagaagcagctctgaccaaagcccttggggaagtggacattcttctgacctggatgcagaaattctacaagctctga

SEQ ID NO: 2 - protein, 206 aa, Artificial sequence
  Synthetic polypeptide
  MISC_FEATURE (1)..(206)
MNFQQRLQSLWTLARPFCPPLLATASQMQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 3 - protein, 157 aa, Artificial sequence
  Synthetic polypeptide
  MISC_FEATURE (1)..(157)
QGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 4 - protein, 103 aa, Artificial sequence
  Synthetic polypeptide
  MISC_FEATURE (1)..(103)
ESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 5 - protein, 24 aa, Artificial sequence
  Synthetic polypeptide
  MISC_FEATURE (1)..(24)
MALWMRLLPLLALLALWGPDPAAA

SEQ ID NO: 6 - DNA, 582 bp, Artificial sequence
  Synthetic polynucleotide
  misc_feature (1)..(582)
atggcgctgtggatgcgcctgctgccgctgctggcgctgctggcgctgtggggcccagatccggcggcggcgcatcaccatcaccatcacgagaacctgtacttccagggcatgcaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggttcctgctattccggagagcattcaaacagttggacgtagaagcagctctgaccaaagcccttggggaagtggacattcttctgacctggatgcagaaattctacaagctctag

SEQ ID NO: 7 - DNA, 567 bp, Artificial sequence
  Synthetic polynucleotide
  misc_feature (1)..(567)
atgacagtgctggcgccagcctggagcccaacaacctatctcctcctgctgctgctgctgagcggatccatgcaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctcgggggttctcatcatcatcatcatcattga

SEQ ID NO: 8 - DNA, 561 bp, Artificial sequence
  Synthetic polynucleotide
  misc_feature (1)..(561)
atgagagcctggatcttttttctgctctgcctcgctggcagagccctggctcatcaccatcaccatcacgagaacctgtacttccagggcatgcaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctga

SEQ ID NO: 9 - DNA, 513 bp, Artificial sequence
  Synthetic polynucleotide
  misc_feature (1)..(513)
atgcagctgctgtcatgcatcgcattgatcttggcgctggtgatgcaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctga

SEQ ID NO: 10 - DNA, 621 bp, Artificial sequence
  Synthetic polynucleotide
  misc_feature (1)..(621)
atgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcagaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctga

SEQ ID NO: 11 - DNA, 582 bp, Artificial sequence
  Synthetic polynucleotide
  misc_feature (1)..(582)
atggcgctgtggatgcgcctgctgccgctgctggcgctgctggcgctgtggggcccagatccggcggcggcgcatcaccatcaccatcacgagaacctgtacttccagggcatgcaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcagaaactaccacaatagaacagttgaagtcaggactctgaagtcattctctactctggccaacaactttgttctcatcgtgtcacaactgcaacccagtcaagaaaatgagatgttttccatcagagacagtgcacacaggcggttcctgctattccggagagcattcaaacagttggacgtagaagcagctctgaccaaagcccttggggaagtggacattcttctgacctggatgcagaaattctacaagctctag

SEQ ID NO: 12 - DNA, 426 bp, Artificial sequence
  Synthetic polynucleotide
  misc_feature (1)..(426)
atggcgctgtggatgcgcctgctgccgctgctggcgctgctggcgctgtggggcccagatccggcggcggcgcatcaccatcaccatcacgagaacctgtacttccagggcatggagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggttcctgctattccggagagcattcaaacagttggacgtagaagcagctctgaccaaagcccttggggaagtggacattcttctgacctggatgcagaaattctacaagctctag

SEQ ID NO: 13 - DNA, 357 bp, Artificial sequence
  Synthetic polynucleotide
  misc_feature (1)..(357)
atgcagctgctgtcatgcatcgcattgatcttggcgctggtgatggagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctga

SEQ ID NO: 14 - DNA, 408 bp, Artificial sequence
  Synthetic polynucleotide
  misc_feature (1)..(408)
atgacagtgctggcgccagcctggagcccaacaacctatctcctcctgctgctgctgctgagcggatccgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctcgggggttctcatcatcatcatcatcattga

SEQ ID NO: 15 - DNA, 471 bp, Artificial sequence
  Synthetic polynucleotide
  misc_feature (1)..(471)
atgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggcggatccgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctga

SEQ ID NO: 16 - DNA, 408 bp, Artificial sequence
  Synthetic polynucleotide
  misc_feature (1)..(408)
atgacagtgctggcgccagcctggagcccaacaacctatctcctcctgctgctgctgctgagcggatccgagagctgttaccttgtccacaccctgctggagttctacttgaaaactgttttcagaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctcgggggttctcatcatcatcatcatcattga

SEQ ID NO: 17 - DNA, 426 bp, Artificial sequence
  Synthetic polynucleotide
  misc_feature (1)..(426)
atggcgctgtggatgcgcctgctgccgctgctggcgctgctggcgctgtggggcccagatccggcggcggcgcatcaccatcaccatcacgagaacctgtacttccagggcatggagagctgttaccttgtccacaccctgctggagttctacttgaaaactgttttcagaaactaccacaatagaacagttgaagtcaggactctgaagtcattctctactctggccaacaactttgttctcatcgtgtcacaactgcaacccagtcaagaaaatgagatgttttccatcagagacagtgcacacaggcggttcctgctattccggagagcattcaaacagttggacgtagaagcagctctgaccaaagcccttggggaagtggacattcttctgacctggatgcagaaattctacaagctctag

SEQ ID NO: 18 - protein, 103 aa, Artificial sequence
  Synthetic polypeptide
  MISC_FEATURE (1)..(103)
ESCYLVHTLLEFYLKTVFRNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 19 - DNA, 63 bp, Artificial Sequence
  synthetic polynucleotide
  misc_feature (1)..(63): Ad.shMDA-9 sense strand
gatccgcggatggcaccaagcattttcaagagaaatgcttggtgccatccgcttttttggaaa

SEQ ID NO: 20 - DNA, 63 bp, Artificial Sequence
  synthetic polynucleotide
  misc_feature (1)..(63): Ad.shMDA-9 antisense strand
agcttttccaaaaaagcggatggcaccaagcatttctcttgaaaatgcttggtgccatccgcg